(19) United States
(12) Patent Application Publication
(10) Pub. No.: US 2005/0234652 A1
(43) Pub. Date: Oct. 20, 2005

(54) Nuclear magnetic resonance-docking of compounds

(75) Inventors: Daniel S. Sem, San Diego, CA (US); Maurizio Pellecchia, San Diego, CA (US)

Correspondence Address: MCDERMOTT, WILL & EMERY, 4370 LA JOLLA VILLAGE DRIVE, SUITE 700, SAN DIEGO, CA, 92122, US

(73) Assignee: Triad Therapeutics, Inc.

(21) Appl. No.: 11/115,850

(22) Filed: Apr. 26, 2005

Related U.S. Application Data
(63) Continuation of application No. 10/158,770, filed on May 30, 2002, now abandoned.
(60) Provisional application No. 60/294,675, filed on May 30, 2001.

(51) Int. Cl. G06F 19/00; G01N 33/48; G01N 33/50

(52) U.S. Cl. 702/19

(57) ABSTRACT

The invention provides a method for determining a structure model for a test ligand bound to a macromolecule binding site. Structural constraints for the test ligand are derived from spectroscopic signals arising from interactions between the test ligand and macromolecule. The structure constraints are used as constraints in docking a structure model of the ligand to a structure model of the macromolecule, or as constraints in overlaying a structure model of the test ligand on the known structure for a reference ligand that binds to the macromolecule. The invention further provides a method for determining a structure model for a macromolecule bound to a ligand. Structural constraints derived from spectroscopically observed interactions of the macromolecule and a reference ligand are used to guide molecular modeling or to evaluate the results of a molecular modeling simulation of the macromolecule.

This application is based on, and claims the benefit of, U.S. Provisional Application No. 60/294,675, filed May 30, 2001, which is incorporated herein by reference.

### BACKGROUND OF THE INVENTION

The present invention relates generally to interactions between macromolecules and ligands and more specifically to Nuclear Magnetic Resonance (NMR) methods for determining structure-related properties of a ligand when bound to a macromolecule.

Structure determination plays a central role in chemistry and biology due to the correlation between the structure of a molecule and its function. Although a full understanding of this correlation is not yet established, one can gain insight into the function of a molecule from its deduced structure. Thus, the structure can provide a strong basis for directing the development of molecules having a desired function. Conversely, the eventual disclosure of a structure for a well studied molecule can have a significant effect in converging apparently disparate observations of function into a consistent description of the molecule's activity.

Practical applications which are becoming increasingly dependent upon structure information include, for example, the production of therapeutic drugs. Structure-based drug design can utilize a three-dimensional structure model of a drug target to predict or simulate interactions with known or hypothetical compounds. Alternatively, in cases where a three-dimensional structure model of a drug target complexed with a ligand is available, therapeutic drugs can be designed to mimic the structural properties of the ligand. Using structure-based methods such as these, lead compounds can be identified for further development.

Screening for lead compounds is another approach that has been used with some success to identify lead compounds for therapeutic targets. Screening involves assaying a library of candidate compounds to identify lead compounds that interact with a drug target. The probability of identifying a lead compound can be increased by providing increased numbers and variety of candidate compounds in the library to be screened. Synthetic methods are available for creating libraries of compounds and include, for example, combinatorial chemistry approaches in which selected chemical groups are variously combined to generate a library of candidate compounds having diverse combinations of the selected chemical groups. In addition, advances have been made to increase the through-put for a number of screening methods. However, for many drug targets the throughput of available screens is prohibitively low. Furthermore, even in cases where high throughput detection is available, limitations on available resources for obtaining a library with sufficient size or diversity, or for obtaining a sufficient quantity of the drug target to support a large screen, can be prohibitive.

The efficiency of library screening approaches can be increased by combining structure-based drug design with the methodologies currently available for library screening. In particular, the probability of identifying a lead compound in a screening approach can be increased by using focused libraries containing member compounds spanning a limited range of desired structural or functional variations. The range of structural or functional variations to be included in a focused library can be determined based on a predicted range of ligand structures obtained from structure-based drug design methods.

For many drug targets of interest, three-dimensional structure models are not presently available. Although methods for structure determination are evolving, it is currently difficult, costly and time consuming to determine the structure of a macromolecule drug target at sufficient resolution to render structure-based drug design practical. It can often be even more difficult to produce a macromolecule-ligand complex in a condition allowing determination of the bound conformation of the ligand. The typically long time period required to obtain structure information useful for developing drug candidates is particularly limiting with regard to exploiting the growing number of potential drug targets identified by genomics research.

Thus, there exists a need for efficient methods to determine the structure of a ligand when bound to a macromolecule for structure-based drug design or for the design of focused libraries of candidate drugs. The present invention satisfies this need and provides related advantages as well.

### SUMMARY OF THE INVENTION

The invention provides a method for determining a structure model for a test ligand bound to a macromolecule binding site, wherein a reference complex can be formed between the macromolecule binding site and a reference ligand, and wherein a test complex can be formed between the macromolecule binding site and a test ligand. The method includes the steps of: (a) identifying reference ligand atoms that are proximal to binding site-localized atoms of the macromolecule in a structure model of the reference complex; (b) observing NMR signals for the reference complex, wherein NMR signals for the binding site-localized atoms and proximal reference ligand atoms interact; (c) assigning NMR signals to the proximal reference ligand atoms in the reference complex; (d) identifying NMR signals for binding site-localized atoms that interact with the assigned NMR signals for the reference ligand atoms; (e) selectively observing pairs of interacting NMR signals for the test complex, each pair including an NMR signal for a test ligand atom that interacts with an NMR signal for a binding site-localized atom identified in part (d); (f) determining distance constraints between test ligand atoms and binding site-localized atoms based on the identified pairs of interacting NMR signals; and (g) docking a structure model of the test ligand to the structure model of the macromolecule binding site based on the distance constraints, thereby determining a structure model for the test ligand bound to the macromolecule binding site.

The invention further provides a method for determining a structure model for a test ligand bound to a macromolecule binding site, wherein a reference complex can be formed between the macromolecule binding site and a reference ligand, and wherein a test complex can be formed between the macromolecule binding site and a test ligand. The method includes the steps of: (a) providing a structure model of the reference ligand bound to the macromolecule binding site; (b) observing NMR signals for the reference complex, wherein NMR signals for reference ligand atoms interact with signals for atoms of the macromolecule; (c) assigning NMR signals to the reference ligand atoms that interact with the atoms of the macromolecule in the reference complex; (d) identifying NMR signals for atoms of the macromolecule that interact with the assigned NMR signals for the reference ligand atoms; (e) selectively observing pairs of interacting NMR signals for the test complex, each pair including an NMR signal for the test ligand that interacts with an NMR signal for an atom of the macromolecule identified in part (d), thereby identifying test ligand atoms and reference ligand atoms that interact with a common macromolecule atom; and (f) overlaying a structure model of the test ligand on the structure model of the reference ligand, wherein atoms for the test ligand and reference ligand that interact with a common macromolecule atom are overlapped, thereby determining a structure model for the test ligand bound to the macromolecule binding site.

The invention provides a method for determining a structure model for a macromolecule binding site, wherein a complex can be formed between the macromolecule binding site and a ligand. The method includes the steps of: (a) observing NMR signals for the complex, wherein NMR signals for ligand atoms interact with signals for atoms of the macromolecule; (b) assigning NMR signals to the ligand atoms that interact with the atoms of the macromolecule in the complex; (c) identifying NMR signals for atoms of the macromolecule that interact with the assigned NMR signals for the ligand atoms; (d) determining the types of amino acids that give rise to the identified NMR signals, thereby determining types of amino acids that are binding site-localized; (e) determining distance constraints between ligand atoms and binding site-localized atoms of the macromolecule; and (f) determining a structure model for the macromolecule binding site based on the sequence of the macromolecule, the type of amino acids that are binding site-localized and the distance constraints.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows in panel A, a structure model of the binding site of DHPR in complex with reference ligands NADH and PDC; in panel B, a 2D (¹³C, ¹H) HMQC spectra of MIT-DHPR; in panels C and D, Met ¹³C^{ε}/¹He^{ε} sub-spectra of MIT-DHPR (black), MIT-DHPR bound to PDC (blue) and MIT-DHPR bound to 4-Cl PDC; and in panel E, a 2D (¹H,¹H) NOESY spectrum of MIT-DHPR bound to NADH and PDC.

FIG. 2 shows in panel A, the structure of nicotinamide mononucleotide (NMNH) test ligand; in panel B, a reference 1D NMR spectrum of NMNH and selective binding site saturated spectrum of NMNH in complex with MIT-DHPR; in panel C, a 2D (¹H,¹H) NOESY spectrum of NMNH in complex with MIT-DHPR; and in panel D, a three-dimensional structure model of the NADH-DHPR crystal complex with NOEs from panel C indicated by dotted lines.

FIG. 3 shows in panel A, the structure of TTM2000_{—}29_{—}85 test ligand; in panel B, a 2D (¹H,¹H) NOESY spectrum of TTM2000_{—}29_{—}85 in complex with MIT-DHPR; and in panel C, a docked structure of TTM2000_{—}29_{—}85 into the three-dimensional X-ray crystal structure model of DHPR.

FIG. 4 shows in panel A, a 2D (¹H,¹H) NOESY spectrum of MIT-DHPR bound to NADH and PDC reference ligands and in panel B, a 2D (¹H,¹H) NOESY spectrum of TTM2000_{—}29_{—}85 test ligand in complex with MIT-DHPR.

FIG. 5 shows a homology structure model for E. coli DOXPR superimposed on the structure model of NAD+ from the X-ray crystal structure model of S. aureas homoserine dehydrogenase.

FIG. 6 shows in panel A, a 2D (¹³C, ¹H) HMQC spectra of MIT-DOXPR; in panel B, a 2D (¹H,¹H) NOESY spectrum of MIT-DOXPR bound to NADP+; in panel C, the met region of a 2D (¹³C, ¹H) HMQC spectra of MIT-DOXPR (blue) and MIT-DOXPR in the presence of Mn²⁺; and in panel D, a 2D (¹H,¹H) NOESY spectrum of a ternary complex formed between MIT-DOXPR, NADPH and a reactive intermediate analog.

FIG. 7 shows the structure of NADH.

### DETAILED DESCRIPTION OF THE INVENTION

The invention provides a method to obtain a three-dimensional model of a ligand bound to a macromolecule by a combination of spectroscopic measurements and computational modeling. Spectroscopic signals arising from ligand-macromolecule interactions in a bound complex can be identified and differentiated from other signals arising from the complex by comparing the spectrum of signals arising from the complex with the spectrum of signals arising from a reference complex. Structure constraints for the ligand are then determined based on the signals identified from the comparison and a structure model of the test ligand bound to the macromolecule is determined by using the structural constraints in a computational molecular modeling process.

An advantage of the invention is that a structure model of a test ligand bound to the macromolecule can be obtained at sufficient resolution to assist in structure-based design of a biologically active agent or drug without the requirement for a complete determination of the structure of the macromolecule-test ligand complex. In particular, by comparing the spectra arising from different complexes, structural constraints for the bound ligand can be obtained without the need to characterize atoms of the macromolecule that do not interact with the ligand. For example, where the spectroscopic method is nuclear magnetic resonance (NMR) spectroscopy, selective observation of magnetic signals arising from ligand-macromolecule interactions allows a structure model of the ligand to be obtained more rapidly than by conventional NMR methods which typically require that resonances be assigned for non-binding site atoms of the macromolecule. Moreover, the methods of the invention can be used with larger macromolecules compared to conventional NMR methods because selective observation of magnetic signals arising from ligand-macromolecule interactions reduces problems associated with resonance overlap.

The invention further provides a method for determining a structure model for a macromolecule bound to a ligand. In the method, structural constraints derived from spectroscopically observed interactions of the macromolecule and ligand are used to guide molecular modeling or to evaluate the results of a molecular modeling simulation. An advantage of the method is that by combining binding site-focused spectroscopic measurements with molecular modeling, an accurate structure model of the macromolecule can be obtained more rapidly and efficiently than with conventional spectroscopic methods.

Definitions

As used herein, the term “structure model” is intended to mean a representation of the relative locations of atoms of a molecule. A representation included in the term can be defined by a coordinate system that is preferably in 3 dimensions, however, manipulation or computation of a model can be performed in 2 dimensions or even 4 or more dimensions in cases where such methods are desired. The location of atoms in a molecule can be described, for example, according to bond angles, bond distances, relative locations of electron density, probable occupancy of atoms at points in space relative to each other, probable occupancy of electrons at points in space relative to each other or combinations thereof. A representation included in the term can contain information for all atoms of a particular molecule or a subset of atoms thereof. Examples of representations included in the term that contain a subset of atoms are those commonly used for polypeptide structures such as ribbon diagrams, and the like, which show the coordinates of the polypeptide backbone while omitting coordinates for all or a portion of the side chain moieties of the polypeptide. Representations for other macromolecules and small molecules included in the term can similarly contain all or a subset of atoms.

A structure model can include a representation that is determined from empirical data derived from, for example, X-ray crystallography or nuclear magnetic resonance spectroscopy. A representation included in the term can also be derived from a theoretical calculation including, for example, comparison to a known structure such as in homology modeling or ab initio molecular modeling. A representation of a structure model can include, for example, an electron density map, atomic coordinates, x-ray structure model, ball and stick model, density map, space filling model, surface map, Connolly surface, Van der Waals surface or CPK model.

As used herein, the term “binding site-localized” is intended to mean an atom of a macromolecule or bound ligand that is proximal to one or more atoms of a second ligand in a complex containing the macromolecule and second ligand or a complex containing the macromolecule and both ligands. Proximal atoms included in the term are those that are within a distance sufficient to cause a chemical interaction such as a hydrogen bond, van der Waals interaction or ionic interaction or to cause a magnetic interaction detectable by a nuclear magnetic resonance spectroscopy measurement used in the methods of the invention. Examples of magnetic effects included in the term are a relaxation effect which can be detected for atoms that are about 10 Å apart or closer, the Nuclear Overhauser Effect which can be detected for atoms that are about 6 < apart or closer or chemical shift due to shielding or de-shielding which can be detected for atoms that are about 10 Å or closer. Atoms that are about 5 Å apart or closer, 4 Å apart or closer, 3 Å apart or closer, 2 Å apart or closer or 1 Å apart or closer are also proximal atoms that are included in the term.

As used herein, the term “macromolecule” is intended to mean a polymeric molecule or complex of polymeric molecules that are associated in solution, including biological and synthetic polymers. Proteins and other polypeptides are particularly useful biological polymers. Other useful biological polymers include polysaccharides and polynucleotides. Polynucleotides are also referred to herein as nucleic acids. Synthetic polymers include plastics and mimetics of biological polymers such as protein-nucleic acids.

As used herein, the term “macromolecule binding site” is intended to mean a portion of a polymeric molecule or complex of polymeric molecules that specifically associates with a ligand. Specific association between a macromolecule and a ligand is understood to be affinity that is characterized by an affinity binding constant (Kₐ) that is 10³ or higher and selectivity such that the macromolecule preferentially binds the ligand over at least one other molecule. A macromolecule that preferentially binds a first ligand over another will have relatively higher affinity for the first ligand such as at least about 2-fold higher affinity for the first ligand compared to the other ligand, at least about 5-fold higher affinity for the first ligand compared to the other ligand, at least about 10-fold higher affinity for the first ligand compared to the other ligand, at least about 20-fold higher affinity for the first ligand compared to the other ligand, at least about 50-fold higher affinity for the first ligand compared to the other ligand or at least about 100-fold higher affinity for the first ligand compared to the other ligand. Accordingly, the term “bound,” when used in reference to a ligand and a macromolecule, is intended to mean specifically associated.

As used herein, the term “complex” is intended to mean a specific non-covalent association between 2 or more molecules. The term can include a reversible association so long as the association is sufficiently stable to be observed by a binding assay.

As used herein, the term “nuclear magnetic resonance (NMR) signal” is intended to mean an output representing the frequency of energy absorbed by a population of magnetically equivalent atoms in a magnetic field, the magnitude of energy absorbed at the frequency by the population and distribution of frequencies around a central frequency. The frequency of energy absorbed by with an atom in a magnetic field can be determined from the location of a peak in an NMR spectrum. The magnitude of energy absorbed at a frequency by a population of atoms can be determined from relative peak intensity. The distribution of frequencies around a central frequency can be determined from the shape of a peak in an NMR spectrum. Accordingly, a collection of nuclear magnetic resonance signals for a molecule or sample containing multiple atoms can be represented in an NMR spectrum, as an atom having a signal of characteristic frequency, intensity and line-shape.

As used herein, the term “nuclear magnetic interaction” is intended to mean an alteration of the nuclear magnetic resonance properties of an atomic nucleus due to a proximal atomic nucleus or at least one electron of a proximal atom. An alteration included in the term can reduce the local magnetic field strength experienced by an atomic nucleus compared to the strength of the field applied to the molecule within which the atom is located which is referred to in the art as shielding. An alteration included in the term can increase the local magnetic field strength experienced by an atomic nucleus compared to the strength of the field applied to the molecule within which the atom is located and is referred to in the art as deshielding. Shielding and deshielding can be observed as changes in chemical shift. An alteration can change the intensity of NMR signals through repopulation of spin states as occurs in the Nuclear Overhauser Effect (NOE). The term can also include an alteration due to a relaxation effect.

As used herein, the term “pair of interacting NMR signals” is intended to mean a first NMR signal and second NMR signal that arise from atomic nuclei that are sufficiently proximal to alter each other's nuclear magnetic resonance properties. A pair of interacting NMR peaks can be represented as a cross-peak in a multidimensional NMR spectrum.

As used herein, the term “ligand” is intended to mean a molecule that can specifically associate with a macromolecule. A molecule included in the term can be a small molecule, a compound or a macromolecule. A molecule included in the term can be naturally occurring such as a DNA, RNA, polypeptide, lipid, carbohydrate, amino acid, nucleotide or hormone or a synthetic molecule or a derivative of a naturally occurring molecule. A derivative can have, for example, an added moiety, a removed moiety or a rearrangement in the relative location of moieties compared to a naturally occurring molecule.

As used herein, the term “reference ligand” is intended to mean a ligand for which one or more structural properties is known or for which a binding site interaction with a macromolecule is known. A structural property included in the term can be a three-dimensional conformation such as a bond angle or relative location of two or more atoms. A three dimensional conformation can be determined at any desired level of resolution sufficient to identify, for example, overall shape of a ligand, identity of individual moieties or identity of individual atoms. The term can include a ligand for which the structure has been partially or completely determined at a particular resolution. A binding site interaction included in the term can be a hydrogen bond, ionic interaction, van der Waals interaction or nuclear magnetic interaction.

As used herein, the term “assigning” is intended to mean correlating a particular NMR signal with a particular atom in a molecule, the atom being defined with respect to atomic number and position in the molecule. The position can be identified as occurring in a particular moiety and at a particular location in a molecule such as at a particular position in the sequence or three dimensional structure of a protein.

As used herein, the term “selectively observing,” when used in reference to a nuclear magnetic resonance signal, is intended to mean preferentially detecting or analyzing a nuclear magnetic resonance signal for an atom in a sample over a nuclear magnetic resonance signal for at least one other atom in the sample. Preferential detection can include enhancing the signal for at least one atom over a signal for another atom or suppressing a signal for at least one atom such that the resolution of a signal for a particular atom is improved. The term can similarly include suppression or enhancement of a particular magnetic interaction. Preferential detection can include detection of signals after application of an NMR pulse sequence such as those described below or detection of isotopically enriched atoms in a macromolecule. Preferential analysis can include omitting one or more magnetic signals or correlations from a spectrum of signals. An example of selective observation includes sparsely labeling a protein and preferentially analyzing a signal that arises from a labeled residue, wherein the labeled residue has been identified based on interactions with a reference ligand in a reference complex containing the protein and reference ligand.

As used herein, the term “distance constraint” is intended to mean a restriction or limit on the length, angle or both length and angle allowed between two atoms in one or more molecular models. A restriction or limit can be a maximum or minimum allowed length or angle that separates at least two atoms or a set of allowed lengths or angles that separate at least two atoms. A set of lengths, angles or both can be used to approximate an area or volume that confines an atom or separates two atoms. A length or angle between atoms can be intramolecular, thereby separating atoms of a molecule, or intermolecular, thereby separating at least one atom of a first molecule, such as a macromolecule, from at least one atom of a second molecule, such as a bound ligand.

As used herein, the term “docking” is intended to mean using a model of a first and second molecule to simulate association of the first and second molecule at a proximity sufficient for at least one atom of the first molecule to be within bonding distance of at least one atom of the second molecule. The term is intended to be consistent with its use in the art pertaining to molecular modeling. A model included in the term can be any of a variety of known representations of a molecule including, for example, a graphical representation of its three-dimensional structure, a set of coordinates, set of distance constraints, set of bond angle constraints or set of other physical or chemical properties or combinations thereof.

As used herein, the term “overlapped,” when used in reference to an atom of a first molecular structure and an atom of a second molecular structure, is intended to mean that the location of the atom of the first molecular structure extends over or covers at least part of the location of the atom of the second molecular structure when the molecular structures are overlaid. Overlap between molecular structures or atoms of the structures can be indicated by a visual comparison and/or computation based comparison.

Docking Structure Models of a Test Ligand and Macromolecule

The invention provides a method for determining a structure model for a test ligand bound to a macromolecule binding site, wherein a reference complex can be formed between the macromolecule binding site and a reference ligand, and wherein a test complex can be formed between the macromolecule binding site and a test ligand. The method includes the steps of: (a) identifying reference ligand atoms that are proximal to binding site-localized atoms of the macromolecule in a structure model of the reference complex; (b) observing NMR signals for the reference complex, wherein NMR signals for the binding site-localized atoms and proximal reference ligand atoms interact; (c) assigning NMR signals to the proximal reference ligand atoms in the reference complex; (d) identifying NMR signals for binding site-localized atoms that interact with the assigned NMR signals for the reference ligand atoms; (e) selectively observing pairs of interacting NMR signals for the test complex, each pair including an NMR signal for a test ligand atom that interacts with an NMR signal for a binding site-localized atom identified in part (d); (f) determining distance constraints between test ligand atoms and binding site-localized atoms based on the identified pairs of interacting NMR signals; and (g) docking a structure model of the test ligand to the structure model of the macromolecule binding site based on the distance constraints, thereby determining a structure model for the test ligand bound to the macromolecule binding site.

The methods can be used to determine a structure model of a bound ligand based on structural constraints obtained from NMR measurements and a known structure model for the macromolecule to which the ligand is bound. Briefly, the structure model is used to assist in assigning resonances for binding site-localized atoms of the macromolecule in a reference complex formed between the macromolecule and a reference ligand. Once resonances for binding site localized atoms of the macromolecule have been assigned, they can be selectively observed for a complex formed between the macromolecule and a test ligand. Based on these selectively observed resonances and their interactions with resonances for the test ligand, distances between the assigned macromolecule atoms and atoms of the ligand can be determined. These distances can then be used as constraints in docking a structure model of the ligand to a structure model of the macromolecule, thereby obtaining a structure model for the bound ligand. This embodiment of the invention is set forth in greater detail below and demonstrated in Example I.

A method of the invention can be used to characterize the structure for a ligand bound to any molecule where the ligand and molecule have atoms that participate in intermolecular interactions that are detectable by NMR methods. The methods of the invention are well suited for characterizing ligands bound to large macromolecules as well as small molecules. The methods are particularly advantageous for use with large macromolecules because selective observation of interactions between a ligand and large macromolecules can provide for more rapid and efficient characterization of ligand structure compared to conventional NMR structure determination which often requires substantially complete assignment of resonances for both the ligand and macromolecule to which it is bound. However, even relatively small molecules for which substantially complete assignment of resonances are possible can be used in the methods of the invention if so desired.

A method of the invention can be performed with a macromolecule and ligand for which binding occurs leading to formation of an NMR detectable complex. Such binding partners can be identified from the scientific literature or by empirical methods. Alternatively, the methods can be used with a relatively uncharacterized test ligand, for example, in a screening application, so long as binding of the ligand to the macromolecule can occur leading to formation of an NMR detectable complex.

Methods of identifying macromolecule-ligand binding partners include, for example, equilibrium binding analysis, competition assays, and kinetic assays as described in Segel, Enzyme Kinetics John Wiley and Sons, New York (1975), and Kyte, Mechanism in Protein Chemistry Garland Pub. (1995). Thermodynamic and kinetic constants can be used to identify and compare macromolecules and ligands that specifically bind each other and include, for example, dissociation constant (K_{d}), association constant (Kₐ) , Michaelis constant (Kₘ) inhibitor dissociation constant (Kᵢₛ) association rate constant (kₒₙ) or dissociation rate constant (k_{off}) . A macromolecule used in a method of the invention can have affinity for a ligand characterized as having a K_{d} of at most 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M or lower. Those skilled in the art will be able to determine the amount or concentration of macromolecule and ligand to include in a sample in order for complex formation to occur using known methods for determining percent occupancy based on equilibrium binding equations, a known or predicted affinity constant of a ligand for a macromolecule and the concentration of the macromolecule in a sample (see, for example, Segel, supra). Alternatively, the amount of macromolecule and ligand to be added can be determined empirically, for example, by titration.

A macromolecule can form a complex with a ligand by specific non-covalent interactions that are reversible, so long as binding is sufficiently stable to produce an NMR detectable complex. Typically, the methods will be used with a macromolecule and ligand that bind to form an inert complex, where neither the ligand or macromolecule undergoes a covalent modification as a result of their interaction with each other. A macromolecule that has enzymatic function can be used in a method of the invention so long as it does not display activity leading to covalent modification of the ligand to which it is bound during the course of acquiring NMR signals. In cases where the macromolecule is a catalyst, a ligand mimetic can be chosen that does not undergo catalysis or that undergoes catalysis at a rate that is slow compared to the timeframe in which ligand interactions are measured. In cases where a reactive ligand is used with an enzyme, conversion of the ligand to a product can be reduced or prevented by altering conditions such that catalytic activity of the enzyme is inhibited. For example, anaerobic conditions can be employed to inhibit. reactions requiring oxygen, pH can be adjusted to inhibit reactions requiring a particular protonation state of a catalytic residue, or a noncompetitive inhibitor can be added.

A method of the invention is well suited for use with large macromolecules because ligands in a complex with a macromolecule can be characterized absent knowledge of the complete structure of the macromolecule or assignment of resonances for a majority of atoms of the macromolecule. In particular, large macromolecules having a monomeric molecular weight greater than 20 kDa, which often are not completely NMR assigned, or for which complete structure models are not available, can be used. Because selective observation of signals arising due to interactions of a macromolecule and bound ligand circumvents complications due to resonance overlap, macromolecules having monomeric molecular weights greater than 25 kDa, 30 kDA, 40 kDa, 50 kDa, 75 kDa, 100 kDa or 150 kDa can be used. Furthermore, a method of the invention can be used with multimeric proteins having at least 2, at least 3, or at least 4 subunits, wherein the subunits have a monomeric molecular weight selected from the range described above.

Because complete NMR assignment of the atoms for a macromolecule is not required to characterize a bound ligand in a method of the invention, a macromolecule can be used for which resonance assignments have not been made for a majority of the atoms in the macromolecule. Thus, a method of the invention can use a macromolecule for which less than 90%, 80%, 70%, 60%, 50%, 40%, 30%, 20% or 10% of the atoms have been assigned a resonance.

Although use of the methods of the invention is exemplified herein with regard to proteins, it is understood that a method of the invention can be used for any other macromolecule that is capable of specifically binding a ligand. Other macromolecules include, for example, biological polymers such as polysaccharides or polynucleotides or synthetic polymers such as plastics and mimetics of biological polymers. A polynucleotide can be, for example, a ribozyme, ribosomal RNA or other RNA that is capable of binding a ligand such as a nucleotide. Non-biological macromolecules such as synthetic polymers and mimetics of biological polymers such as protein nucleic acids can also be used in a method of the invention.

A macromolecule can be isolated for use in the methods from a native tissue or organism, from a population of cells maintained in culture, or from a recombinant organism or cell culture. Methods for isolating a protein are known in the art and are described, for example, in Scopes, Protein Purification: Principles and Practice, 3^{rd} Ed., Springer-Verlag, New York (1994); Duetscher, Methods in Enzymology, Vol 182, Academic Press, San Diego (1990); and Coligan et al., Current protocols in Protein Science, John Wiley and Sons, Baltimore, Md. (2000).

A macromolecule can be cloned and expressed in a recombinant organism using methods that are known to those skilled in the art including, for example, polymerase chain reaction (PCR) and other molecular biology techniques (Dieffenbach and Dveksler, eds., PCR Primer: A Laboratory Manual, Cold Spring Harbor Laboratory Press, Plainview, N.Y. (1995); Sambrook et al., Molecular Cloning: A Laboratory Manual, 2nd ed., Cold Spring Harbor Laboratory Press, Plainview, N.Y. (1989); Ausubel et al., Current Protocols in Molecular Biology, Vols. 1-3, John Wiley & Sons (1998)). The gene or cDNA encoding the macromolecule is cloned into an appropriate expression vector for expression in an organism such as bacteria, insect cells, yeast or mammalian cells.

Appropriate expression vectors include those that are replicable in eukaryotic cells and/or prokaryotic cells and can remain episomal or be integrated into the host cell genome. Suitable vectors for expression in prokaryotic or eukaryotic cells are well known to those skilled in the art as described, for example, in Ausubel et al., supra. Vectors useful for expression in eukaryotic cells can include, for example, regulatory elements including the SV40 early promoter, the cytomegalovirus (CMV) promoter, the mouse mammary tumor virus (MMTV) steroid-inducible promoter, Moloney murine leukemia virus (MMLV) promoter, and the like. A vector useful in the methods of the invention can include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are known in the art as described, for example, in Sambrook et al., supra (1989) and Ausubel et al., supra (1998). One skilled in the art will know or can readily determine an appropriate promoter for expression in a particular host cell.

If desired, a protein can be expressed as a fusion with an affinity tag that facilitates purification and detection of the protein. For example, a protein can be expressed as a fusion with a poly-His tag, which can be purified by metal chelate chromatography. Other useful affinity purification tags which can be expressed as fusions with the target protein and used to affinity purify the protein include, for example, a biotin, polyhistidine tag (Qiagen; Chatsworth, Calif.), antibody epitope such as the flag peptide (Sigma; St Louis, Mo.), glutathione-S-transferase (Amersham Pharmacia; Piscataway, N.J.), cellulose binding domain (Novagen; Madison, Wis.), calmodulin (Stratagene; San Diego, Calif.), staphylococcus protein A (Pharmacia; Uppsala, Sweden), maltose binding protein (New England BioLabs; Beverley, Mass.) or strep-tag (Genosys; Woodlands, Tex.) or minor modifications thereof.

The invention can be used with any ligand that binds with a macromolecule to form a complex including, for example, chemical or biological molecules such as simple or complex organic molecules, metal-containing compounds, carbohydrates, peptides, peptidomimetics, carbohydrates, lipids, nucleic acids, and the like.

In one embodiment, the methods of the invention can be used with a ligand that is a nucleotide derivative including, for example, a nicotinamide adenine dinucleotide-related molecule. Nicotinamide adenine dinucleotide-related (NAD-related) molecules that can be used in the methods of the invention can be selected from the group consisting of oxidized nicotinamide adenine dinucleotide (NAD⁺), reduced nicotinamide adenine dinucleotide (NADH), oxidized nicotinamide adenine dinucleotide phosphate (NADP⁺), and reduced nicotinamide adenine dinucleotide phosphate (NADPH). An NAD-related molecule can also be a mimetic of the above-described molecules.

A mimetic is a molecule that has at least one function that is substantially the same as a function of a second molecule including, for example, the function of binding to the same macromolecule as the second molecule. A mimetic of a ligand can be identified according to its ability to bind to the same sites on a macromolecule as the ligand. For example, a mimetic can be identified by a binding competition assay using a ligand and a mimetic. The structure of a mimetic can be similar or different compared to the structure of the second molecule, so long as they bind competitively to the same macromolecule. A mimetic can be a molecule having portions similar to corresponding portions of the ligand in terms of structure or function.

Examples of mimetics to the common ligand NADH, for example cibacron blue, are described in Dye-Ligand Chromatography, Amicon Corp., Lexington Mass. (1980). Numerous other examples of NADH-mimetics, including useful modifications to obtain such mimetics, are described in Everse et al. (eds.), The Pyridine Nucleotide Coenzymes, Academic Press, New York N.Y. (1982). Particular analogs include nicotinamide 2-aminopurine dinucleotide, nicotinamide 8-azidoadenine dinucleotide, nicotinamide 1-deazapurine dinucleotide, 3-aminopyridine adenine dinucleotide, 3-acetyl pyridine adenine dinucleotide, thiazole amide adenine dinucleotide, 3-diazoacetylpyridine adenine dinucleotide and 5-aminonicotinamide adenine dinucleotide. Particular mimetics can be identified and selected by ligand-displacement assays, for example using competitive binding assays with a known ligand as is known in the art. Mimetic candidates can also be identified by searching databases of compounds for structural similarity with the common ligand or a mimetic.

In another embodiment, the methods of the invention can be used with a ligand that is an adenosine phosphate-related molecule. Adenosine phosphate-related molecules can be selected from the group consisting of adenosine triphosphate (ATP), adenosine diphosphate (ADP), adenosine monophosphate (AMP), and cyclic adenosine monophosphate (cAMP). An adenosine phophate-related molecule can also be a mimetic of the above-described molecules. A mimetic of an adenosine phosphate-related molecule that can be used in the invention includes, for example, quercetin, adenylylimidodiphosphate (AMP-PNP) or olomoucine.

A ligand useful in the methods of the invention can be a cofactor, coenzyme or vitamin including, for example, NAD, NADP, or ATP as described above. Other examples include thiamine (vitamin B₁), riboflavin (vitamin B₂), pyridoximine (vitamin B₆), cobalamin (vitamin B₁₂), pyrophosphate, flavin adenine dinucleotide (FAD), flavin mononucleotide (FMN), pyridoxal phosphate, coenzyme A, ascorbate (vitamin C), niacin, biotin, heme, porphyrin, folate, tetrahydrofolate, nucleotide such as guanosine triphosphate, cytidine triphosphate, thymidine triphosphate, uridine triphosphate, retinol (vitamin A), calciferol (vitamin D₂), ubiquinone, ubiquitin, α-tocopherol (vitamin E), farnesyl, geranylgeranyl, pterin, pteridine or S-adenosyl methionine (SAM).

A polypeptide can be used as a ligand in the invention. For example, a ligand can be a naturally occurring polypeptide ligand such as a ubiquitin or polypeptide hormone including, for example, insulin, human growth hormone, thyrotropin releasing hormone, adrenocorticotropic hormone, parathyroid hormone, follicle stimulating hormone, thyroid stimulating hormone, luteinizing hormone, human chorionic gonadotropin, epidermal growth factor, nerve growth factor and the like. In addition a polypeptide ligand can be a non-naturally occurring polypeptide that has binding activity. Such polypeptide ligands can be identified, for example, by screening a synthetic polypeptide library such as a phage display library or combinatorial polypeptide library. A polypeptide ligand can also contain amino acid analogs or derivatives such as those described below.

A nucleic acid can also be used as a ligand in the invention. Examples of nucleic acid ligands useful in the invention include DNA, such as genomic DNA or cDNA or RNA such as mRNA, ribosomal RNA or tRNA. A nucleic acid ligand can also be a synthetic oligonucleotide. Such ligands can be identified by screening a random oligonucleotide library for ligand binding activity. Nucleic acid ligands can also be isolated from a natural source or produced in a recombinant system using well known methods in the art including, for example, those described above with respect to macromolecule nucleic acids.

A ligand used in the invention can be an amino acid, amino acid analog or derivatized amino acid. An amino acid ligand can be one of the 20 essential amino acids or any other amino acid isolated from a natural source. Amino acid analogs useful in the invention include, for example, neurotransmitters such as gamma amino butyric acid, serotonin, dopamine, or norepenephrine or hormones such as thyroxine, epinephrine or melatonin. A synthetic amino acid, or analog thereof, can also be used in the invention. A synthetic amino acid can include chemical modifications of an amino acid such as alkylation, acylation, carbamylation, iodination, or any modification that derivatizes the amino acid. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. Naturally occurring amino acid derivatives of the twenty standard amino acids can also be included in a cluster of bound conformations including, for example, 4-hydroxyproline, 5-hydroxylysine, 3-methylhistidine, homoserine, ornithine or carboxyglutamate.

A lipid ligand can also be used in the invention. Examples of lipid ligands include triglycerides, phospholipids, glycolipids or steroids. Steroids useful in the invention include, for example, glucocorticoids, mineralocorticoids, androgens, estrogens or progestins.

Another type of ligand that can be used in the invention is a carbohydrate. A carbohydrate ligand can be a monosaccharide such as glucose, fructose, ribose, glyceraldehyde, or erythrose; a disaccharide such as lactose, sucrose, or maltose; oligosaccharide such as those recognized by lectins such as agglutinin, peanut lectin or phytohemagglutinin, or a polysaccharide such as cellulose, chitin, or glycogen.

A reference complex used in a method of the invention can be a previously observed molecular structure acquired, for example, by searching a database of existing structures. An example of a database that includes structures of macromolecule-ligand complexes is the Protein Data Bank (PDB, operated by the Research Collaboratory for Structural Bioinformatics, see Berman et al., Nucleic Acids Research, 28:235-242 (2000)). A database can be searched, for example, by querying based on chemical property information or on structural information. In the latter approach, an algorithm based on finding a match to a template can be used as described, for example, in Martin, “Database Searching in Drug Design,” J. Med. Chem. 35:2145-2154 (1992).

A reference complex can be obtained from an empirical measurement, or from a database. Data specifying a three-dimensional structure model can be acquired using any method available in the art for structural determination of a ligand bound to a polypeptide. For example, X-ray crystallography can be performed with a crystallized complex of a polypeptide and ligand to determine binding site-localized atoms of the macromolecule that are proximal to a ligand. Methods for obtaining such crystal complexes and determining structures from them are well known in the art as described, for example, in McRee et al., Practical Protein Crystallography, Academic Press, San Diego 1993; Stout and Jensen, X-ray Structure Determination: A practical guide, 2^{nd} Ed. Wiley, New York (1989); and McPherson, The Preparation and Analysis of Protein Crystals, Wiley, New York (1982). Another method useful for determining a bound conformation of a ligand bound to a polypeptide is Nuclear Magnetic Resonance (NMR). NMR methods are well known in the art and include those described for example in Reid, Protein NMR Techniques, Humana Press, Totowa N.J. (1997); and Cavanaugh et al., Protein NMR Spectroscopy: Principles and Practice, ch. 7, Academic Press, San Diego Calif. (1996). A reference complex can also be obtained from homology modeling using a structure-based alignment algorithm such as the MODELER module in MSI Insight II (Sali and Blundell, J. Mol. Biol. 234:779-815 (1993)) or PrISM (Yang and Honig Proteins 37:66-72 (1999)).

A molecular structure can be conveniently stored and manipulated using structural coordinates. Structural coordinates can occur in any format known in the art so long as the format can provide an accurate reproduction of the observed structure. For example, crystal coordinates can occur in a variety of file types including, for example, .fin, .df, .phs, or .pdb as described for example in McRee, supra. Although the examples above describe structural coordinates derived from X-ray crystallographic analysis or NMR spectroscopy, one skilled in the art will recognize that structural coordinates can be derived from any method known in the art to determine a bound conformation of a ligand bound to a protein. Furthermore, a structure model of a bound ligand can be determined without structurally characterizing the macromolecule to which it is bound using, for example, transferred NOEs as described in Roberts, Curr. Opin. Biotech. 10:42-47 (1999).

Any representation that correlates with the structure of a macromolecule-ligand complex can be used to evaluate a reference complex or to model a binding interaction in the methods of the invention. For example, a convenient and commonly used representation is a displayed image of the structure. Displayed images that are particularly useful for determining the bound conformation of a ligand bound to polypeptides include, for example, ball and stick models, density maps, space filling models, surface map, Connolly surfaces, Van der Waals surfaces or CPK models. Display of images as a computer output, for example, on a video screen can be advantageous, for example, in computational docking and overlay methods, as described below.

Structures at atomic level resolution can be useful in the methods of the invention. Resolution, when used to describe molecular structures, refers to the minimum distance that can be resolved in the observed structure. Thus, resolution where individual atoms can be resolved is referred to in the art as atomic resolution. Resolution is commonly reported as a numerical value in units of Angstroms (Å, 10⁻¹⁰ meter) correlated with the minimum distance which can be resolved such that smaller values indicate higher resolution. Bound conformations of a ligand useful in the methods of the invention can have a resolution with a value that is at most about 10 Å including, for example, at most about 5 Å, 3 Å, 2.5 Å, 2.0 Å, 1.5 Å, 1.0 Å, 0.8 Å, 0.6 Å, 0.4 Å, or 0.2 Å or better. Resolution can also be reported as an all atom root mean square deviation (RMSD) as used, for example, in reporting NMR data. Bound conformations of a ligand useful in the methods of the invention can have an all atom RMSD between multiple calculated structures with a value that is at most about 10 Å including, for example, at most about 5 Å, 3 Å, 2.5 Å, 2.0 Å, 1.5 Å, 1.0 Å, 0.8 Å, 0.6 Å, 0.4 Å, or about 0.2 Å or better.

Binding-site localized atoms in a reference structure model of a macromolecule-ligand complex can be identified based on proximity of the residues to the ligand. Proximity can be determined as a distance separating two atoms that is sufficient for a particular interaction to occur. For example, in NMR applications proximity can be determined as a distance between an atom of the ligand and an atom of the macromolecule within which magnetic interactions can occur between the two atoms. When the interaction is a magnetic relaxation effect or a chemical shift effect, proximal atoms can be identified as those that are separated by at most about 10 Å. Proximity as determined for an NOE interaction is within at most about 6 Å. Proximity can also be based on the distance within which chemical interactions occur such as a hydrogen bond which, depending upon the atoms involved, is about 3 Å; an ionic bond which, depending upon the atoms involved, is about 3 Å or a van der Waals interaction which, depending upon the atoms involved, is about 3 Å to 4 Å. Those skilled in the art can readily determine, for any particular pair of identifiable atoms in a structure model of a reference complex, whether or not the atoms are sufficiently proximal for the above described interactions to occur based on known or predictable properties of each atom. Accordingly. proximal atoms can be identified as those that are separated from each other by at most about 9 Å, 8 Å, 7 Å, 6 Å, 5 Å, 4 Å, 3 Å, or 2 Å.

Interactions between binding site-localized atoms of a macromolecule and a bound ligand can give rise to a variety of interacting NMR signals that can be used in the methods of the invention to determine the conformation of the bound ligand. The Nuclear Overhauser Effect (NOE) can cause detectible changes in the NMR signal of an atom that is proximal to a perturbed atom and can be measured, for example, using 3D HSQC-NOESY. The signal changes are the result of magnetization transfer to the proximal atom. Since an NOE occurs by spatial proximity, not merely connection via chemical bonds, it is especially useful for identifying molecules that interact in a complex. Furthermore, the strength of an NOE between proximal atoms can be correlated with distance between the atoms as described, for example, in Neuhaus et al. “The Nuclear Overhauser Effect in Structural and Conformational Analysis”, Wiley-VCH, New York, 2000. As described in further detail below and demonstrated in the Examples, intramolecular distances or intermolecular distances derived from NOE signals can be used to determine a structural model of a ligand bound to a macromolecule.

Other interacting signals that can be detected in a method of the invention include, for example, a chemical shift perturbation, or a relaxation effect. A through space interaction between a first atom and a proximal atom can cause the resonance signal for the first atom to shift upfield or downfield due to shielding or deshielding effects, respectively, of the proximal atom. Accordingly, an interaction between a binding site-localized atom of a macromolecule and an atom of a bound ligand can cause a chemical shift perturbation where the resonance for either atom is shifted compared to its resonance in the absence of the other atom. Chemical shift effects are distance dependent and can be used to determine inter-atomic distances as described, for example, in Wishart and Case, Methods in Enzymology 338:3-34 (2001).

A through space interaction between a binding site-localized atom of a macromolecule and an atom of a bound ligand can cause transfer of energy between the atoms resulting in a detectable change in the rate of relaxation. Thus, a change in the rate of relaxation, for example, due to a spin-lattice or T₁ relaxation effect can be used in a method for determining a structure model of a ligand bound to a macromolecule. Relaxation effects are distance dependent and can be used to estimate interatomic distances. The use of relaxation effects to determine distance between atoms is described, for example, in Battiste and Wagner, Biochem. 39:5355-5365 (2000); Jacob et al., Biophys. J. 77:1086-1092 (1999). An equation describing the distance dependence of relaxation effects is described in Saunders and Hunter, “Modern NMR Spectroscopy” p167 (1987).

Information on the interactions between a macromolecule and ligand can be obtained using heteronuclear NMR experiments. Heteronuclear NMR experiments are particularly useful with larger proteins as described in Cavanaugh et al., Protein NMR Spectroscopy: Principles and Practice, ch. 7, Academic Press, San Diego Calif. (1996). For example, double resonance methods, also referred to as two-dimensional NMR methods, can measure the chemical shifts of two types of nuclei. A well established 2-D method is the ¹H-¹⁵N heteronuclear single quantum coherence (HSQC) experiment. Another method is the heteronuclear multiple quantum. coherence (HMQC) experiment. Numerous other variant experiments and modifications are known in the art including nuclear Overhauser enhancement spectroscopy experiments (NOESY), for example NOE experiments involving a {¹H, ¹H} NOESY step. Interacting NMR signals that arise from atoms of a ligand that interact with atoms of a macromolecule can be identified from cross-peaks in a two-dimensional NMR spectrum, or in higher dimensional spectra, as set forth below. Two-dimensional and three-dimensional methods can also be used to obtain assignments for binding site localized atoms of a macromolecule using sequential assignment methods.

Higher-dimensional NMR methods can often eliminate problems with cross peak overlap if spectra are too crowded and can be used to observe magnetic interactions of additional types of nuclei or to make assignments based on these additional types of nuclei. In particular, the NMR method used can correlate ¹H, ¹³C and ¹⁵N (Kay et al., J. Magn. Reson. 89:496-514 (1990); Grzesiek and Bax, J. Magn. Reson. 96:432-440 (1992)), for example, in an HNCA experiment. Other heteronuclear NMR methods can be used including, for example, HNCO, HNCACB, CBCA(CO)NH, HBHA(CO)CA, HN(CO)CA, H(CA)NH, H(CC){TOCSY}NH, and heteronuclear resolved NOESY. Particular multidimensional techniques for identifying compounds that bind to target molecules are described in U.S. Pat. No. 5,698,401 to Fesik et al., and U.S. Pat. No. 5,804,390 to Fesik et al. Related publications include PCT publications WO 97/18469, WO 97/18471 and WO 98/48264. However, these techniques, sometimes described as “SAR by NMR,” require the complete determination of the three-dimensional structure of the enzyme (Shuker et al., Science 274:1531-1534 (1996); Hajduk et al., J. Am. Chem. Soc. 119:5818-5827 (1997)). In contrast, the methods of the invention do not require determining the complete structure of the macromolecule; instead, it rapidly provides sufficient information to obtain structure constraints for a bound ligand which are used in a computational modeling method and subsequent determination of a structure model for the bound ligand.

With the appropriate sample requirements and isotope filtered experiments, cross-correlations, cross-relaxations and residual dipolar couplings can be measured and provide structural information. A macromolecule can be isotopically labeled with ²H atoms to simplify spectra by replacing NMR-visible ¹H atoms, with ¹⁵N or ¹³C to enrich the macromolecule for these NMR visible isotopes, or with a combination of these atom isotopes. For example, ²H atoms can be incorporated at both exchangeable and non-exchangeable positions in a macromolecule by growing an organism expressing the macromolecule in the presence of D₂O (²H₂O). ²H atoms can be incorporated or maintained at exchangeable positions, such as at amides or hydroxyls of a protein, by carrying out steps in the isolation of the macromolecule in deuterated solvent. For protein labeling, acetate or glucose can be provided as the sole carbon source in the presence of D₂O if complete deuteration on carbon is desired. If pyruvate is used as the sole carbon source, there will be protons only on the methyl groups of Ala, Val, Leu and Ile (Kay, Biochem. Cell Biol. 75:1-15 (1997). Labeling with ¹⁵N can be achieved by growing an organism expressing a macromolecule of interest in an ¹⁵N-containing nitrogen source such as salts of ¹⁵NH₄⁺ like (¹⁵NH₄)₂SO₄ or ¹⁵NH₄Cl.

A polymeric macromolecule can be labeled by providing isotopically enriched monomers, or precursors thereof, to the growth medium of a production organism. Incorporation of an amino acid having a particular position labeled, such as a backbone or side chain position, can be achieved by supplementing the growth medium of the production organism with the labeled amino acid or with a labeled precursor of the amino acid. Using methods such as those demonstrated in Example I a protein can be labeled at the methyl positions of methionine, isoleucine and threonine. Selective side chain 13C/1H labeling of Val, Tyr, Phe, Trp and His can be achieved using conditions described in Goto et al., Curr. Opin. Struct. Biol. 10:585-592 (2000). Similarly, nucleic acids and polysaccharides can be labeled with isotopically enriched nucleotides or saccharides, respectively. These and other related methods for isotopically labeling macromolecules have been described previously (Laroche, et al., Biotechnology 12:1119-1124 (1994); LeMaster Methods Enzymol. 177:23-43 (1989); Muchmore et al., Methods Enzymol. 177:44-73 (1989); Reilly and Fairbrother, J. Biomolecular NMR 4:459-462 (1994); Ventors et al., J. Biomol. NMR 5:339-344 (1995); and Yamazaki et al., J. Am. Chem. Soc. 116:11655-11666 (1994)).

In addition, homonuclear and heteronuclear two and three bond J couplings can be obtained to provide information on torsion angles (Wuthrich, supra). For example, torsion angles can be measured and distinguished by measuring the three bond ³¹p-¹³C4′ J coupling constants that correspond to torsion angles of bound NADPH ligands (Marino, Acc. Chem. Res. 32:614-623 (1999)). Basically, two ¹H-¹³C correlation spectra can be obtained with and without ³¹P decoupling during ¹³C evolution. The intensity ratio of the ¹H 4′/¹³C4′ cross peak from each spectra is proportional to the ³¹P-¹³C4′ J coupling constant for the bound NADPH. Those skilled in the art will recognize that similar methods can be extended to other bound ligands by using an appropriate correlation experiment to observe the desired two or three bond system.

NMR signals can be assigned to binding site-localized atoms of a macromolecule by comparing, for macromolecule-ligand complexes of different composition, the signals that arise due to magnetic interactions between the macromolecule and ligand. The signals that differ between the different complexes are identified as potentially arising from binding site-localized atoms of the macromolecule. These signals can be assigned to a specific amino acid in the macromolecule structure based on the binding site-localized atoms identified in the reference macromolecule structure model.

Signals arising from binding site-localized atoms can be identified by comparing NMR spectra for a macromolecule in the presence and absence of a ligand. The comparison can be facilitated by using a labeled macromolecule, especially if the macromolecule is relatively large. For example, as demonstrated in Example I, the ¹³C^{ε}/¹H^{ε} resonances of DHPR Met17 were assigned due to the change in chemical shift upon binding of PDC.

Often ligand binding, in addition to causing chemical shift in binding site-localized atoms due to interactions with the ligand, causes chemical shift changes due to intra-molecular magnetic interactions of a macromolecule. In this case, chemical shifts due to interactions between binding site-localized atoms and a ligand can be identified by a differential chemical shift method in which the spectra of the target protein bound to two slightly different ligands are compared. Methods for determining a binding site of a protein based on differential chemical shifts for a series of closely related ligands is described in Medek et al., J. AM. Chem. Soc. 122:1241-1242 (2000).

Thus, a method of the invention can further include a step of detecting NMR signals for a second reference complex including a second reference ligand bound to the macromolecule binding site, wherein the second reference ligand is a mimetic of the first reference ligand, and identifying NMR signals for binding site localized atoms by comparing the NMR signals detected in a first reference complex with the NMR signals detected in the second reference complex. A signal for a binding site-localized atom can be identified due to differential chemical shift for interactions with a moiety of a first ligand compared to a second ligand where the moiety is altered or absent. The identification of a signal for a binding site-localized atom can also be made based on the loss or gain of resonances in a spectra for a first complex compared to a second complex.

Assignment or identification of NMR signals in a method of the invention can be facilitated by sparsely labeling the macromolecule at particular types of atoms or residues or selectively labeling binding site residues where possible. Prominent signals arising due to interactions between the labeled residues and a bound ligand can be identified or assigned. If a protein binding site contains an amino acid that is unique compared to the rest of the protein sequence or if the binding site contains an amino acid that is in relatively low abundance in the rest of the protein, the amino acid can be assigned based on its being relatively uniquely labeled and observation of an interaction with the ligand. For example, sparse labeling can be used in combination with observation of chemical shifts to identify binding site-localized atoms of a large macromolecule. As demonstrated in Example I, when sparsely labeled DHPR (MIT-DHPR) binds to PDC by contrast to the ‘chemically perturbed’ variant, 4-Cl PDC, distinct changes in chemical shift for only one of the methionine ¹³C^{ε}/¹H^{ε} resonances was detected, thereby indicating that the chemically shifted signals were associated with Met17.

In the case of a kinase, a first NMR spectra can be obtained in the presence of ATP and a second in the presence of ADP. Differences in the two spectra due to binding site localized atoms that interact with the Υ-phosphate of ATP can be identified. Based on properties of the signals that differ between the two spectra such as the chemical shift for the binding site-localized atoms and based on the identities of binding site-localized atoms of a reference kinase structure model that are consistent with these properties the signal can be assigned. In another example, in the case of a NAD binding protein such as a dehydrogenase, the NAD molecule can be modified, for example, by separately binding adenine mononucleotide or nicotinamide mononucleotide. Changes in the spectra obtained in the presence of either ligand can be observed and compared to the reference dehydrogenase structure model used to assign resonances for the binding site-localized atoms. In either of the above cases, sparse labeling can be used to make particular residues more prominent in the NMR spectra and facilitate the differential chemical shift approach.

Signals can also be assigned by titrating a ligand and monitoring progressive changes in chemical shifts or peak intensity. Titration can be used in combination with difference spectra methods in which two or more ligands are used. For example, in order to determine which signals arising from a complex with a first ligand correspond to shifted or absent cross peaks in a complex with a second ligand, it is possible to titrate one or both ligands and monitor progressive changes in chemical shifts or peak intensity.

A method of the invention can include comparing spectra for complexes that differ by containing different variants of the macromolecule bound to the same ligand. In particular, a method of the invention can further include a step of detecting NMR signals for a second reference complex including the reference ligand bound to a variant macromolecule binding site and identifying NMR signals for binding site localized atoms by comparing the NMR signals detected in a first reference complex with the NMR signals detected in the second reference complex. The variant binding site can be produced by mutation to substitute a particular monomer, such as an amino acid or nucleotide, for another or by chemical modification of a particular monomer. A combination of mutation and chemical modification can also be used, such as by mutating a chemically inert amino acid to replace it with an amino acid that is reactive toward a particular modifying agent and subsequently modifying the mutated amino acid.

The residues to be changed can be selected based on the binding site-localized atoms identified from the structure model of the reference complex. Mutants can be made using known methods of site directed mutagenesis as described for example in Sambrook et al., supra (1989) and Ausubel et al., supra (1998). A signal for a binding site-localized atom can be identified due to the loss of resonances in a spectra for a complex where the atom is absent compared to a complex in which the atom is present.

Another way to obtain resonance assignments for binding site-localized atoms is by measuring NOEs between atoms of the macromolecule and atoms of the ligand. Given the resonance assignments of a reference ligand, which are easily obtained with conventional 1D and 2D NMR experiments, assignments of binding site-localized atoms in a macromolecule-ligand complex can be obtained by structurally mapping them relative to protons of the reference ligand. The atoms of a ligand can be perturbed through either a selective inversion of its resonances using radio-frequency pulses wherein a transient Nuclear Overhauser Effect is observed or the ligand atoms can be perturbed by a complete saturation of its resonances using radio-frequency pulses, wherein a steady-state NOE is observed as described, for example, in Neuhaus et al., “The Nuclear Overhauser Effect in Structural and Conformational Analysis,” Wiley-VCH, New York pp 129-279 (2000). Thus, binding site-localized atoms are mapped according to their proximity to the different protons on a reference ligand. The use of NOEs to identify binding site-localized atoms is demonstrated in Example I where binding site residues of DHPR are mapped relative to bound NADH or PDC.

Once signals for binding site-localized atoms of a macromolecule have been assigned, the signals arising therefrom can be monitored to determine if a candidate ligand binds to the macromolecule. Thus, the invention provides a method of identifying a ligand that binds to a macromolecule. The method can include the steps of (a) identifying reference ligand atoms that are proximal to binding site-localized atoms of the macromolecule in a structure model of the reference complex; (b) observing NMR signals for the reference complex, wherein NMR signals for the binding site-localized atoms and proximal reference ligand atoms interact; (c) assigning NMR signals to the proximal reference ligand atoms in the reference complex; (d) identifying NMR signals for binding site-localized atoms that interact with the assigned NMR signals for the reference ligand atoms; (e) selectively observing pairs of interacting NMR signals for a test complex formed by a candidate ligand and the macromolecule; and (f) identifying a candidate ligand that interacts with the macromolecule to form a pair of interacting NMR signals, the pair including an NMR signal for a test ligand atom that interacts with an NMR signal for a binding site-localized atom identified in part (d), as a ligand for the macromolecule.

Signals for binding site-localized atoms of a macromolecule once identified can be used to determine affinity of a ligand for a macromolecule. For example, a ligand can be titrated into a sample containing the macromolecule and the relative amount of complex formed at each concentration of ligand can be determined by observing changes in a particular signal that has been identified as binding site-localized. The binding affinity can then be determined by fitting the results to a binding equation using known methods as described, for example, in Segel, supra (1975), and Kyte, supra (1995). In contrast to previously described NMR-based methods for determining affinity, such as SAR by NMR (Shuker et al., Science 274:1531-4 (1996)), assignment of residues is not necessary in order to determine ligand affinity.

A method of the invention can include a step of selectively observing pairs of interacting NMR signals for a test complex, each pair including an NMR signal for a test ligand atom that interacts with an assigned NMR signal for a binding site-localized atom. Once signals for binding site-localized atoms of a macromolecule have been assigned, a complex can be formed between the macromolecule and a test ligand and interactions between the binding site-localized atoms and the test ligand selectively observed. These pairs of interacting signals can be selectively observed over NMR signals that arise from non-binding site-localized atoms of the macromolecule. Because a large portion of the atoms of a macromolecule are generally non-binding site-localized, the pairs of signals are often selectively observed over at least 50%, 60%, 70%, 80%, or 90% of the atoms in the macromolecule. Even for smaller macromolecules where a smaller portion of the atoms are binding site-localized, the pairs of signals can be selectively observed over at least 10%, 20%, 30%, or 40% of the atoms in the macromolecule.

Interactions between the binding site-localized atoms and the test ligand can be selectively observed by selective acquisition of signals arising from the assigned binding site-localized atoms in the presence of the test ligand. Selective acquisition of signals for the assigned binding site-localized atoms can be achieved using an appropriate pulse sequence such as SEA-TROSY which allows selective observation of exchangeable protons such as those that are surface-localized and binding-site localized as described, for example, in Pellecchia et al., J. Am. Chem. Soc. 123:4633 (2001). Selective observation can also be achieved by sparse labeling of particular atoms or residues using methods such as those described above and demonstrated in the Examples.

Interactions between a macromolecule and a test ligand can also be selectively observed by selectively analyzing the signals arising from the assigned binding site-localized atoms. Thus, analysis of interacting signals can focus on cross-peaks that are formed between assigned resonances of the macromolecule and resonances of the test ligand while analysis of other resonances that are due to non-binding site-localized atoms can be deferred or avoided. Thus, for large macromolecules analysis of a majority of the signals arising from its atoms, and peaks in the resulting spectrum, can be deferred or avoided, thereby making structure analysis more rapid and efficient.

The distance between binding site-localized atoms of the macromolecule and atoms of the test ligand can be measured from the strength of the magnetic interactions between them. The strength of the magnetic interactions can be determined, for example, from the intensity of an NOE signal between two atoms because the strength of an NOE interaction between two protons is dependent on 1/r⁶, where r is the distance between the two protons. For example, the distance between atoms can be estimated based on measurement of NOE build-up rates as described, for example, in Neuhaus et al., supra (2000). Since T₁ relaxation effects have a 1/r⁶ dependence on distance as does NOE, such relaxation effects can be used to measure distance, particularly between paramagnetic species and NMR-active nuclei such as protons (Battiste and Wagner, supra (2000); Jacob et al., supra (1999) and Saunders and Hunter, supra (1987)). Also shielding and deshielding effects of atoms on NMR-active nuclei have distance and directionality dependence that can be used in computational structure determination (Wishart and Case, supra (2001)).

NMR signals arising from a ligand, such as a test ligand, when bound to a macromolecule in a complex, can be observed in a method of the invention, thereby providing structural information for the ligand that can be used as structural constraints in a modeling step of the method. In a fast exchange regime, cross-correlated relaxation measurements can provide structural information on ligand torsion angles (Carlomagno et al., J. Am. Chem Soc. 121:1945-1948 (1999)). These measurements include the ¹H-¹H dipole-dipole cross-correlation but can be extended to other cross-correlated relaxation mechanisms involving also homonuclear and heteronuclear chemical shielding anisotropy relaxation, as well as quadrupolar relaxation. For most of these heteronuclear experiments, the natural abundance of the isotope can be exploited. In cases where natural abundance of the isotope measured is not sufficient, isotope enriched ligands can be obtained from commercial sources such as Isotek (Miamisburg, Ohio) or Cambridge Isotope Laboratories (Andover, Mass.) or prepared by methods known in the art. Another method to determine a conformation of a ligand in a fast exchange regime is use of residual homonuclear and heteronuclear dipolar couplings in partially aligned samples (Tolman et al. Proc. Natl. Acad. Sci. USA 92:9279-9283 (1995)).

In the slow exchange regime, the NMR signals arising from the bound conformation of the ligand are distinguished from those of the macromolecule to which it is bound in order to reduce resonance overlap. This can be achieved with different isotope labeling schemes of macromolecule, ligand or both. For large systems, perdeuteration of macromolecules and TROSY-type experiments (Pervushkin, Proc. Natl. Acad. Sci. USA 94:12366-12371 (1997)) can be used to minimize signal losses due to fast transverse relaxation of the resonances of the complex. Methods utilizing a TROSY pulse sequence can be further simplified using a SEA-TROSY pulse sequence as described, for example, in Pellecchia et al., J. Am. Chem. Soc. 123:4633 (2001).

The distances measured between atoms of a macromolecule and atoms of a test ligand can be used as distance constraints in docking a structure model of a test ligand into a structure model of a macromolecule binding site. Molecular docking explores the binding modes of two interacting molecules, depending on their topographic features or energetic interactions, and aims to fit them into conformations that lead to favorable interactions. It therefore constitutes a useful step in determining the active conformation of a drug or inhibitor as described, for example, in Doucet and Weber, “Computer-Aided Drug Design” Academic Press (1996). In cases where docking is performed with a structure model of a macromolecule-reference ligand complex, the coordinates for the reference ligand can be removed by editing the file containing the structure coordinates for the complex. The edited file can be used for docking simulations such that the test ligand is docked into the macromolecule binding site lacking the reference ligand.

NMR-derived distance constraints can be used to dock the structures using distance geometry, torsion angle dynamics, simulated annealing or a molecular dynamics or molecular mechanics algorithm. Such methods are described for example, in Crippen and Havel “Distance Geometry and Molecular Conformation,” John Wiley and Sons (1988). Docking a macromolecule and ligand using NMR-derived distance constraints in distance geometry and torsion angle dynamics approaches can be performed, for example, using the DYANA computer algorithm, Guntert et al., J. Mol. Biol. 273:283 (1997). Other algorithms available in the art for fitting a ligand structure to a binding site include, for example, DOCK (Kuntz et al., J. Mol. Biol. 161:269-288 (1982)) and INSIGHT II (Molecular Simulations Inc., San Diego, Calif.). A three dimensional model of the docked macromolecule and test ligand can subsequently be energy minimized using standard force fields using methods described, for example, in Doucet and Weber, supra (1996).

To take into account eventual protein conformational rearrangement upon binding, molecular dynamics simulation can then be performed, and intra-molecular NOEs between NMR-active nuclei in the protein can also be measured, identified and included in the simulation. In addition, constraints from residual dipolar coupling, coupling through a hydrogen bond, chemical shift effects or relaxation effects can be included in a structure calculation.

Overlaying Structure Models for a Test Ligand and Reference Ligand

The invention further provides a method for determining a structure model for a test ligand bound to a macromolecule binding site, wherein a reference complex can be formed between the macromolecule binding site and a reference ligand, and wherein a test complex can be formed between the macromolecule binding site and a test ligand. The method includes the steps of: (a) providing a structure model of the reference ligand bound to the macromolecule binding site; (b) observing NMR signals for the reference complex, wherein NMR signals for reference ligand atoms interact with signals for atoms of the macromolecule; (c) assigning NMR signals to the reference ligand atoms that interact with the atoms of the macromolecule in the reference complex; (d) identifying NMR signals for atoms of the -macromolecule that interact with the assigned NMR signals for the reference ligand atoms; (e) selectively observing pairs of interacting NMR signals for the test complex, each pair including an NMR signal for the test ligand that interacts with an NMR signal for an atom of the macromolecule identified in part (d), thereby identifying test ligand atoms and reference ligand atoms that interact with a common macromolecule atom; and (f) overlaying a structure model of the test ligand on the structure model of the reference ligand, wherein atoms for the test ligand and reference ligand that interact with a common macromolecule atom are overlapped, thereby determining a structure model for the test ligand bound to the macromolecule binding site.

A method of the invention can be used to obtain a structure model for a bound ligand by comparison to the structure for a bound reference ligand but without a need to perform docking simulations of the ligand to the macromolecule. Thus, knowledge of a structure model of the macromolecule to which the ligands bind is not necessary. Briefly, NMR signals are identified as arising from binding site-localized atoms of a macromolecule based on interactions of the signals with signals from a reference ligand. In this embodiment assignment of the identified signals to a particular atom of the macromolecule is not necessary. Once signals for binding site localized atoms of the macromolecule have been identified, they can be selectively observed for a complex formed between the macromolecule and a test ligand. An identified signal that interacts with both an atom of the reference ligand and an atom of the test ligand can be identified as arising from a binding site-localized atom that is proximal to both ligand atoms. A structure model for the test ligand can be overlaid on a structure model for the reference ligand such that atoms that interact with the same macromolecule-derived signal are overlapped, thereby obtaining a structure model for the bound test ligand. This embodiment of the invention is set forth in greater detail below and demonstrated in Example II.

A method incorporating a step of overlaying ligands can be performed using any macromolecule and ligand for which binding occurs leading to formation of an NMR detectable complex, as set forth above. A macromolecule or ligand can be obtained using the methods described above or any of a variety of methods known in the art.

A structure model for a reference ligand bound to a macromolecule can be obtained from the sources set forth above including, for example, an X-ray crystal structure, NMR structure model, or theoretical model. Because a structure model of the macromolecule is not required, a structure model for a reference ligand that is to be used in an overlay method of the invention can be obtained using a method that determines the bound ligand structure while solving the structure of the macromolecule only partially or not at all. Thus, NMR methods, such as those described above for distinguishing ligand signals over those from the macromolecule to which it is bound, can be used. A particularly useful method for determining the structure of a ligand when bound to a macromolecule is measurement of transferred NOEs as described in Roberts, supra (1999).

NMR signals for a ligand-macromolecule complex can be observed using the methods described above. However, assignment of the observed signals to a particular atom of the macromolecule is not necessary. Rather, identification that an observed signal arises from a binding site localized atom of a macromolecule is sufficient. Such an identification can be made by observing differences in chemical shift or peak intensity for signals arising from a macromolecule in the presence or absence of a reference ligand. This method of identification can be carried out in a titration mode where progressive changes in chemical shift or peak intensity are monitored as a reference ligand is titrated into a sample containing the macromolecule. Those peaks which undergo a change in intensity or chemical shift that are ligand concentration dependent are candidates for being due to binding site-localized atoms of the macromolecule. Similarly, the resonances arising from the ligand can be assigned, and those signals from the macromolecule that interact with the ligand resonances, for example, as NOE cross-peaks, can be identified as candidates for being due to binding site-localized atoms of the macromolecule. Similarly, spectra for complexes that differ by being bound to different ligands can be compared. A signal for a binding site-localized atom can be identified due to differential chemical shift or loss or gain of resonances in a spectra for a first complex compared to a second complex.

Once signals arising from binding site-localized atoms in a reference complex that interact with atoms of a reference ligand have been identified, the distance between each pair of interacting atoms, one from the macromolecule and one from the reference ligand, can be determined. The distance can be determined using the methods set forth above, such as measurements based on NOE intensity.

A complex can be formed between a test ligand and the same macromolecule that was included in a reference complex. Signals that were identified as arising from binding site-localized atoms of the macromolecule and their interactions with the test ligand can be selectively observed using the methods set forth above. The distance between each pair of interacting atoms, one from the macromolecule and one from the test ligand, can also be determined as set forth above.

A structure model for a test ligand bound to a macromolecule can be obtained by overlaying a structure model of the test ligand on a structure model of a reference ligand bound to the macromolecule. The ligands can be overlaid such that pairs of atoms, one from each ligand, that are proximal to the same atom of the macromolecule are constrained based on their distances from the atom of the macromolecule. In formulating such a constraint, the atoms from the reference ligand and the test ligand are considered to approach the atom of the macromolecule from the same direction due to the steric constraints present in typical macromolecule binding sites. By setting the directions from the two ligand atoms to the atom of the macromolecule as coincident, the constraint on the two ligand atoms relative to each other when overlaid can be based on the difference in the two ligand macromolecule interatomic distances. For example, if a test ligand atom is 6 Å from a binding site-localized atom and a reference ligand atom is 5 Å from the binding site-localized atom, then a constraint in overlaying the two ligand atoms can be based on a 1 Å difference in location. Two structures can be overlaid using a distance geometry or related algorithm such as the OVERLAY routine in INSIGHT II (Molecular Simulations Inc., San Diego Calif.).

In cases where a three-dimensional structure model is available for the binding site to which a reference ligand and test ligand bind, a structure model for the bound test ligand can be obtained by a combination of the overlay and docking methods described above. The overlay and docking simulations can be carried out sequentially, for example, by first obtaining a test ligand structure model by overlaying with a reference ligand followed by docking the test ligand structure model into the binding site structure model. Such methods can also be carried out iteratively until a structure model for the test ligand having desired properties is obtained.

A structure model of a bound conformation of a test ligand obtained by the methods of the invention can include all of the atoms of the test ligand or a portion of the atoms. A structure model of a portion of a ligand can include selected atoms or bonds of a ligand and can include, for example, a continuous sequence of atoms or bonds or a discontinuous sequence of selected atoms or bonds that, when described independent of the complete ligand structure, may not appear to be attached to each other. Those skilled in the art will understand that either a complete or partial structure of a ligand can be valuable in designing a drug or inhibitor that targets a macromolecule. For example, a partial structure can be used to search a database of structures or to guide in synthesis of a compound or library of compounds as is commonly done with pharmacophore models.

A structure model of a ligand bound to a macromolecule can be used to design a binding compound that is specific for the macromolecule. The model, even if partial with respect to all of the atoms in the ligand, can be used as a scaffold or set of constraints for developing a compound having enhanced binding affinity or specificity for the macromolecule. Using similar methods a ligand structure model can be used to design a combinatorial synthesis producing a library of compounds having properties consistent or similar to the model which can be then be screened for enhanced binding affinity or specificity for the macromolecule. An algorithm can be used to design a binding compound based on a ligand structure model including, for example, LUDI as described by Bohm, J. Comput. Aided Mol. Des. 6:61-78 (1992).

A structure model of a ligand can also be used to explore the binding mode of the ligand to a macromolecule using a 3D-QSAR (quantitative structure activity relationship) approach. 3D-QSAR approaches can be used to optimize ligand affinity by searching for favorable interactions based on considerations of binding energy and steric interactions as described, for example, in Cramer et al., J. Am. Chem. Soc. 110:5959 (1988) and Greco et al., J. Computer Aided Molecular Design 8:97 (1994).

A method of the invention can also be used in the design of a bi-ligand compound inhibitor of a macromolecule that binds two ligands in adjacent binding sites. One or both of the ligands that bind to adjacent sites of a macromolecule can be structurally characterized in a method of the invention and a linker designed using NMR-SOLVE. The NMR-SOLVE method can be used to identify proximal ligands and measure the distance between the ligands without the need to structurally characterize the macromolecule to which they are bound, as described in U.S. Pat. No. 6,333,149. Based on the distance measured between adjacent ligands in a ternary complex using NMR-SOLVE and structural characterization of one or both ligands using a method of the present invention locations for a linker on each ligand can be determined as well as the length of the linker to join the two ligands such that both can bind to their respective binding sites when linked as a bi-ligand. The use of NMR-SOLVE in a method of the invention for obtaining a bi-ligand is demonstrated in Example IV.

Validating a Macromolecule Structure Model

The invention provides a method for determining a structure model for a macromolecule binding site, wherein a complex can be formed between the macromolecule binding site and a ligand. The method includes the steps of: (a) observing NMR signals for the complex, wherein NMR signals for ligand atoms interact with signals for atoms of the macromolecule; (b) assigning NMR signals to the ligand atoms that interact with the atoms of the macromolecule in the complex; (c) identifying NMR signals for atoms of the macromolecule that interact with the assigned NMR signals for the ligand atoms; (d) determining the types of amino acids that give rise to the identified NMR signals, thereby determining types of amino acids that are binding site-localized; (e) determining distance constraints between ligand atoms and binding site-localized atoms of the macromolecule; and (f) determining a structure model for the macromolecule binding site based on the sequence of the macromolecule, the type of amino acids that are binding site-localized and the distance constraints.

A method of the invention can be used to determine a structure model for a binding site of a macromolecule based on structural constraints obtained from NMR measurements and a known structure model for the ligand to which the macromolecule is bound. Briefly, NMR signals are identified as arising from binding site-localized atoms of a macromolecule based on interactions of the signals with signals from a reference ligand. In this embodiment the identified signals are assigned to an atom in a type of monomer present in the macromolecule, such as an amino acid in a protein or nucleotide in a nucleic acid. However, the location of the particular monomer in the sequence of the macromolecule need not be known. Based on these selectively observed resonances and their interactions with resonances for the ligand, distances between the monomers of the macromolecule and atoms of the ligand can be determined. These distances can then be used as constraints in the conformation of the macromolecule that reduce the solution space for determining the structure of the macromolecule in a computational algorithm. The method can be performed as demonstrated in Example III.

A method for determining a structure model for a macromolecule binding site can be performed using any macromolecule and ligand for which binding occurs leading to formation of an NMR detectable complex, as set forth above. A macromolecule or ligand can be obtained using the methods described above or any of a variety of methods known in the art. A structure model for a reference ligand bound to a macromolecule can be obtained from the sources set forth above including, for example, an X-ray crystal structure, NMR structure model, or theoretical model.

NMR signals for a ligand-macromolecule complex can be observed using the methods described above. However, assignment of the observed signals to an atom of a monomer at a particular location in the sequence or structure of the macromolecule is not necessary. Rather, identification that an observed signal arises from an atom in a particular type of binding site localized monomer of a macromolecule is sufficient. Such an identification can be made by observing differences in chemical shift or peak intensity for signals arising from a macromolecule in the presence or absence of a reference ligand. This method of identification can be carried out in a titration mode where progressive changes in chemical shift or peak intensity are monitored as a reference ligand is titrated into a sample containing the macromolecule. Those peaks which undergo a change in intensity or chemical shift that are ligand concentration dependent are candidates for being due to binding site-localized atoms of the macromolecule. Similarly, the resonances arising from the ligand can be assigned, and those signals from the macromolecule that interact with the ligand resonances, for example, as NOE cross-peaks, can be identified as candidates for being due to atoms in binding site-localized monomers of the macromolecule. Similarly, spectra for complexes that differ by being bound to different ligands can be compared. A signal for a binding site-localized atom can be identified due to differential chemical shift or loss or gain of resonances in a spectra for a first complex-compared to a second complex.

Once signals arising from binding site-localized monomers in a reference complex that interact with a ligand have been identified, the distance between each pair of interacting atoms, one from the macromolecule and one from the ligand, can be determined. The distance can be determined using the methods set forth above, such as measurements based on NOE intensity.

The distances determined from interactions observed between a monomer of a macromolecule and a ligand can be used in combination with a computational process of determining a structure model of the macromolecule. A variety of methods are known in the art for modeling the three dimensional structure of a macromolecule such as a protein according to its sequence of monomers and a structure of a homologous macromolecule used as a template. A template macromolecule can be identified based on structural or functional similarities using methods known in the art. Structural similarity can be identified, for example, by sequence analysis at the nucleotide or amino acid level. One method for determining if two macromolecules are related is BLAST, Basic Local Alignment Search Tool. (available on the internet at ncbi.nlm.nih.gov/BLAST/; administered by The National Center for Biotechnology Information, Bethesda Md.). BLAST is a set of similarity search programs designed to examine all available sequence databases and can function to search for similarities in protein or nucleotide sequences. A BLAST search provides search scores that have a well-defined statistical interpretation. Furthermore, BLAST uses a heuristic algorithm that seeks local alignments and is therefore able to detect relationships among sequences which share only isolated regions of similarity (Altschul et al., J. Mol. Biol. 215:403-410 (1990)).

In addition to the originally described BLAST (Altschul et al., supra, 1990), modifications to the algorithm have been made (Altschul et al., Nucleic Acids Res. 25:3389-3402 (1997)). One modification is Gapped BLAST, which allows gaps, either insertions or deletions, to be introduced into alignments. Allowing gaps in alignments tends to reflect biologic relationships more closely. A second modification is PSI-BLAST, which is a sensitive way to search for sequence homologs. PSI-BLAST performs an initial Gapped BLAST search and uses information from any significant alignments to construct a position-specific score matrix, which replaces the query sequence for the next round of database searching. A PSI-BLAST search is. often more sensitive to weak but biologically relevant sequence similarities.

Another resource that can be used to identify a template macromolecule is PROSITE. (Available on the internet at expasy.ch/sprot/prosite.html; administered by The Swiss Institute for Bioinformatics, Switzerland). PROSITE is a method of determining the function of uncharacterized proteins translated from genomic or cDNA sequences (Bairoch et al., Nucleic Acids Res. 25:217-221 (1997)). PROSITE consists of a database of biologically significant sites and patterns that can be used to identify which known family of proteins, if any, the new sequence belongs. In some cases, the sequence of an unknown protein is too distantly related to any protein of known structure to detect its resemblance by overall sequence alignment. However, related proteins can be identified by the occurrence in its sequence of a particular cluster of amino acid residues, which can be called a pattern, motif, signature or fingerprint. PROSITE uses a computer algorithm to search for motifs that identify proteins as family members. PROSITE also maintains a compilation of previously identified motifs, which can be used to determine if a newly identified protein is a member of a known protein family.

Yet another resource for identifying a homologous sequence that is useful as a template in a structure modeling algorithm is Structural Classification of Proteins (SCOP, Available on the internet at scop.mrc-lmb.cam.ac.uk/scop/, administered by Medical Research council, Cambridge, England. (which is incorporated herein by reference). SCOP maintains a compilation of previously determined protein tertiary folds from which structural comparison, at a priomary sequence or tertiary level, can be made to identify protein family members having similar motifs (Murzin et al., J. Mol. Biol. 247:536-540 (1995)).

A template macromolecule can be selected based on a conserved and recognizable primary sequence motif. A template macromolecule can also be recognized based on similar function. A protein family can be identified based on the ability of its members to bind a natural common ligand that is already known. For example, it is known that dehydrogenases bind to dinucleotides such as NAD or NADP. Therefore, NAD or NADP are natural common ligands to a number of dehydrogenase family members. Similarly, kinases bind ATP, which is therefore a natural common ligand to kinases.

Once a sufficiently homologous template macromolecule is chosen, for which a three-dimensional structure model is available, homology modeling can be carried out using an algorithm such as the MODELER module in MSI Insight II (Sali and Blundell, supra (1993)) or PrISM (Yang and Honig, supra (1999)). If desired, visualization tools can be used to assist with homology modeling. Available visualization tools include, for example, GRASP (Nicholls, A., supra), ALADDIN (Van Drie et al., J. Comput. Aided Mol. Des. 3:225-51 (1989)), INSIGHT II (Molecular Simulations Inc., San Diego Calif.), RASMOL (Sayle et al., Trends Biochem Sci. 20:374-376 (1995)) or MOLMOL (Koradi et al., J. Mol. Graphics 14:51-55 (1996)). Construction of a homology model for a protein based on a template identified by the sequence homology is demonstrated in Example III.

A method for determining a structure model for a macromolecule binding site can include a step of determining a structure model for the macromolecule binding site using an ab initio algorithm that is constrained based on the sequence of the macromolecule, the type of amino acids that are binding site-localized and the distance constraints. A computational process can be performed to determine a structure of the macromolecule of interest where various combinations of monomers, that are of the type identified as binding site-localized, are constrained to be located proximal to each other. The proximity of the monomers, whether amino acids in a protein or nucleotides in a nucleic acid, can be constrained to dimensions that are consistent with the set of distances measured for the macromolecule-ligand complex. The methods can be performed iteratively to test various combinations of positionaly-defined monomers, that are of the type identified as binding site-localized, for the ability to produce a satisfactory three-dimensional structure model of the macromolecule.

Alternatively, a homology model can be computed without initially considering the constraints derived from NMR observation of the ligand-macromolecule complex. The constraints can then be used to determine if the structure model is satisfactory. If a model is not satisfactory, as judged by producing a binding site that is not consistent with the NMR-observed constraints, the modeling process can be repeated, iteratively, or a new modeling approach used until a more satisfactory model is obtained.

A three dimensional structure model of a macromolecule determined by the methods of the invention can be useful for identifying a function of the macromolecule. For example, residues of a protein that are involved in binding can be identified using a model of the invention. Residues identified as participating in binding can be modified, for example, to engineer new functions into a protein, to reduce an intrinsic activity of a protein, or to enhance an intrinsic activity of a protein. In another example, a model of a protein can be compared to other protein structures to identify similar functions. Exemplary functions that can be identified from a protein structure include binding interactions with other protein and catalytic activities.

The following examples are intended to illustrate but not limit the present invention.

### EXAMPLE I

### Docking of a Furoic Acid-Based Inhibitor into the Binding Site of DHPR

This Example demonstrates determination of a three dimensional model of a furoic acid-based inhibitor bound to the NADH binding site of E. coli Dihydrodipicolinate reductase (DHPR). In particular, this example describes, expression and purification of isotopically labeled DHPR; NMR measurements of a DHPR-NADH complex to assign DHPR binding site residues that interact with NADH; NOE measurements of a DHPR-inhibitor complex to determine distances between the binding site residues and the inhibitor; and docking of the inhibitor to a previously determined structure model of DHPR based on distance constraints derived from the NOE measurements.

A. Expression of Isotopically Labeled DHPR

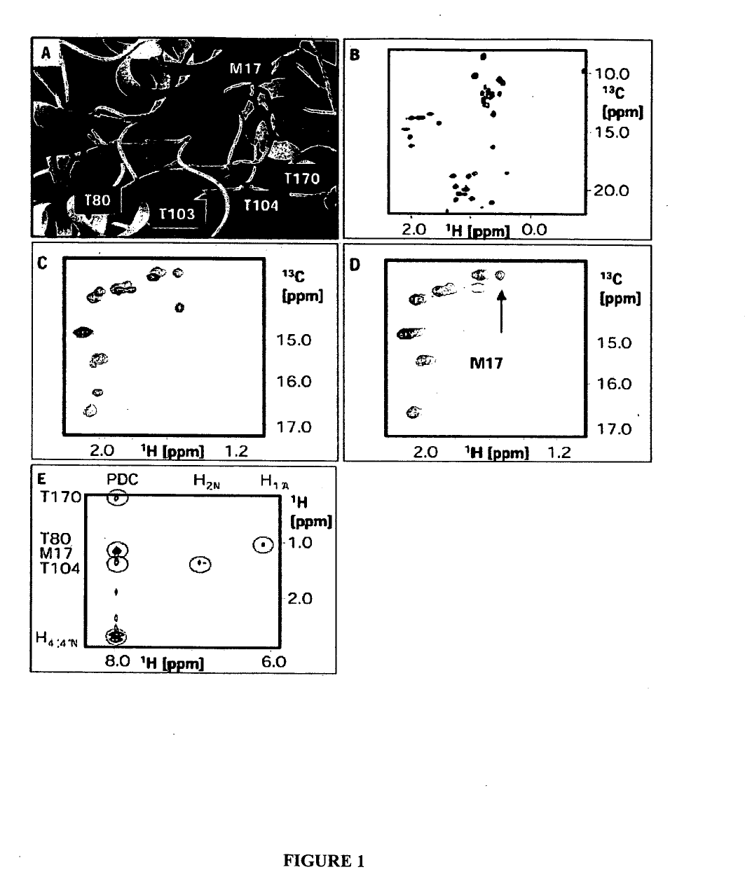

E. coli DHPR was selectively labeled with ¹³C^{ε}/¹H Met, ¹³C^{δ}/¹H Ile and ¹³C/¹H Thr and uniformly labeled with ²H. The resulting labeled protein is referred to as MIT-DHPR. This labeling scheme was chosen based on analysis of the three-dimensional X-ray structure of the enzyme (Scopin et al., Biochem. 36:15081-15088 (1997), PDB code larz) which revealed that several threonine residues (T80, T103, T104 and T170) occur in both the binding site for the NADH cofactor and the binding site for the substrate ligand as shown in FIG. 1A. A methionine residue (M17) is also present at the interface of these binding sites. Specific labeling of particular residue types, in this case methionine, isoleucine and threonine, has the advantage of simplifying 2D NMR spectra. Furthermore, narrow line widths can be obtained because of the fast rotation of methyl protons. Labeling methyl protons provides the added advantage of increased sensitivity because of the presence of three equivalent protons. As shown in FIG. 1B, all of the expected cross-peaks were clearly observed and resolved in the 2D (¹³C, ¹H) correlation spectrum of MIT-DHPR.

The nucleic acid encoding E. coli DHPR in pET11a (Novagen) was obtained by PCR amplification from the E. coli DHPR gene and the amplified product was subcloned into pET21a+ (Novagen) at the NdeI and BamH1 sites to produce the pET11a+/DHPR vector. E. coli DHPR was expressed from BL21 (DE3) Gold E. coli (Stratagene) that had been transduced with the pET11a+/DHPR vector.

E. coli containing the pET11a+/DHPR vector was conditioned to grow on deuterated medium by 50 fold dilution of the cells from a starter culture (LB, 100 μg/mL carbenicillin, OD₆₀₀ about 0.4 to 0.5) into M9 minimal media containing 90% D₂O; growth to an OD₆₀₀ of about 0.3 to 0.4; subsequent 40 fold dilution into M9 minimal media containing 100% D₂O, uniformly ²H-enriched D-glucose and uniformly ¹⁵N-enriched ammonium chloride; and overnight incubation. The conditioned culture was diluted 20 fold into 100 mL of the latter M9 minimal media, incubated with shaking in a 1 L baffled flask for about 16 hours (final OD₆₀₀ of about 4.5-5.0), and the 100 mL culture was used to inoculate 1 L basal fermentation media containing 2 g/L ²H-D-glucose and 0.8 g/L ¹⁵NH₄Cl and 0.5× trace metal and nutrient solution.

The 1 L culture was incubated in a BioFlo 3000 fermentor (New England Biolabs) with pH of the culture maintained at 7.0 through the automated feeding of 0.1 N NaOD and aeration through continuous sparging with dried air. The culture was grown until the pH was stable and the dissolved oxygen level began to rise, at which time a batch feed solution consisting of 3 g/L ²H-D-glucose, 1.2 g/L ¹⁵NH₄Cl, 0.5×trace metal and nutrient solution, and 100 mg U-¹H/¹⁵N/¹³C-labeled threonine was added. After a re-equilibration period of 10-15 minutes, DHPR expression was induced by addition of 2 mM IPTG and allowed to proceed until the pH feed was inactive and the pH value began to rise (final cell densities were about OD₆₀₀ 0.4-0.5). Cells were collected by centrifugation and frozen at −80° C.

Isotopically labeled reagents were obtained from commercial sources including Martek Biosciences Corp., Cambridge Isotope Laboratories or Isotec, Inc. Other reagents were obtained from commercial sources unless indicated otherwise. The M9 minimal media was adapted from Metzler et. al., J. Am. Chem. Soc., 118:6800-6801 (1996) and contained 5 g/L D-glucose, 2 g/L NH₄Cl, 10.725 g/L Na₂HPO₄.H2O, 4.5 g/L KH₂HPO₄, 0.75 g/L NaCl, 2mM MgSO₄ and 2 μL of a 1000× trace metal and nutrient solution (2 mg/mL CaCl₂, 2 mg/mL ZnSO₄.7H₂O, 15 mg/mL thiamine, 10 mg/mL niacinamide, 1 mg/mL biotin, 1 mg/mL choline chloride, 1 mg/mL pantotenic acid, 1 mg/mL pyridoxine, 1 mg/mL folic acid, 10.8 mg/mL FeCl₃.6H₂O, 0.7 mg/mL Na₂MoO₄.2H₂O, 0.8 mg/mL CuSO₄.2H₂O and 0.2 mg/mL H₃BO₃) .

B. Purification of Isotopically Labeled DHPR

The labeled DHPR protein was isolated using the following steps carried out at 4° C. Cell pellets were resuspended in lysis buffer (50 mM Tris pH 7.5, 100 mM NaCl, 1 mM EDTA, and 1 mL protease inhibitor cocktail (Sigma #P8465)) by homogenization (IKAWORKS Ultraturax model T25 homogenizer) and lysed by passage through a microfluidizer (3×18,000 psi, Microfluidics model 110Y). Insoluble cellular debris was removed by centrifugation at 20,000×g, for 45 minutes. The resulting supernatant was dialysed against 50 mM Tris pH 7.8, 1 mM EDTA and subsequently cleared via centrifugation at 20,000×g for 45 minutes. The resulting supernatant was fractionated using Fast Flow Q-SEPHAROSE™ (Pharmacia) equilibrated in 25 mM Tris pH 7.8, 1 mM EDTA, and eluted with a 0 to 1 M NaCl gradient. Fractions containing DHPR were identified by SDS-PAGE, pooled, loaded onto a Blue Sepharose 6 Fast Flow (Pharmacia) column equilibrated in 20 mM Tris pH 7.8, 1 mM EDTA, and eluted with equilibration buffer containing 2 M NaCl, yielding greater than 99% pure DHPR.

DHPR-mutants M17I and T104S were produced by site directed mutagenesis of the pET11a+/DHPR plasmid using the QUICKCHANGE™ Site-Directed Mutagenesis Kit (Stratagene). DHPR-mutants were expressed and purified essentially as described above. Mutants are identified by the convention known in the art where, for example, M17I refers to mutation of DHPR leading to removal of methionine and replacement with Isoleucine at position 17.

C. NMR Measurements

NMR measurements were performed on a Bruker DRX700 spectrometer operating at 700 MHz ¹H frequency and equipped with a triple resonance probe and a triple axis gradient coil. Samples contained about 75 micromolar DHPR (300 micromolar monomer), in 25 mM TrisD₁₁ in D₂O buffer, pH=7.8 and were maintained at 303° K. during the measurements. The sample volume was 0.15 ml in shigemi tubes. Protein-ligand complexes were prepared by slowly adding to a protein solution 2.5 microliters of DMSO-D₆ solution containing 30 to 100 mM ligand.

Based on the large chemical shift difference of Thr ¹³C^{Υ} (about 18 ppm) and ¹³C^{β (about} 70 ppm), selective WURST adiabatic decoupling during the ¹³C evolution was implemented to decouple ¹³C^{Γ} from ¹³C^{β}, resulting in line narrowing in the Thr ¹³C^{Γ} dimension. This line narrowing dramatically reduced the overlap among the fourteen ¹³C/¹H^{Γ} resonances in labeled DHPR. This effect was apparent in the 2D HMQC spectrum where Thr ¹³C^{Γ}/¹H^{Γ} cross-peaks were significantly narrower than those corresponding to Ile ¹³O/¹H^{δ}. Typically each 2D (¹³C,¹) spectrum was recorded in about 30 minutes.

A HMQC magnetization transfer can be used as an alternative to the HSQC scheme because, based on theoretical principles, the ¹H-¹³C dipole-dipole relaxation mechanism, responsible for the fast ¹³C transverse relaxation rates, will be largely attenuated Cavenaugh et al., supra (1996). In uniformly labeled protein samples, HSQC sequences exhibit better relaxation properties than HMQC due to strong dipole-dipole relaxation between protons introduced during the heteronuclear evolution time. The selectively labeled samples, however, will be mostly deuterated and proton-proton dipole-dipole interactions can occur (in this particular case) only between Met, Thr and Ile residues. As Thr and Met residues are usually not clustered and also not part of the hydrophobic core of proteins, these dipole-dipole interactions are small, hence HMQC is preferred in this case.

Typical 2D (¹H,¹H) NOESY spectra (Anil-Kumar et al., Biochim. Biophys. Res. Comm. 95:1-6 (1980)) were acquired with 256×2048 complex points and with mixing times between 50 ms and 500 ms. Thr ¹³C^{δ} decoupling during t1 evolution was achieved with a ¹³C 180 degree refocusing pulse. ¹³C decoupling during the acquisition was achieved with a GARP composite decoupling sequence (Shaka et al. J. Magn. Reson. 64:547-552 (1985)). The measuring time for a 2D (¹H,¹H) NOESY varied from about 12 h to 48 h, depending on the ligand concentration (between 0.5 mM to 2 mM). Eventual ambiguities due to proton overlap among Thr and Met residues were resolved by recording a 3D (¹³C,¹H) resolved (¹H, ¹H) NOESY measurement (Fesik et al., J. Magn. Reson. 78:588-593 (1988)). QUIET NOESY (Quenching Undesirable Indirect External Trouble in NOESY, Neuhaus et al. “The Nuclear Overhauser Effect in Structural and Conformational Analysis”, Wiley-VCH, New York, 2000) measurements were also performed to avoid artificial NOE cross-peaks arising from spin diffusion. These measurements differ from a conventional NOESY measurements by the presence in the middle of the mixing time of a selective (or a combination of selective) 180 degree pulse(s) to invert only the signals of the two protons for which the length of separation is to be determined. Several REBURP selective pulses were implemented for this purpose.

D. Assigning DHPR Binding Site Residues

The resonance assignments for DHPR residues Thr80, Thr104 and Met17 were obtained as follows. Differential chemical shift perturbation was observed by comparing the spectra of MIT-DHPR bound to 2,6-pyridinedicarboxylate (PDC) and the spectra of MIT-DHPR bound to 4-Cl PDC. Distinct changes in chemical shift for only one of the methionine ¹³C^{ε}/¹H^{ε} resonances was detected, which therefore identified the signals as being associated with M17 as shown in FIG. 1D. Both PDC and 4-Cl PDC bound to DHPR with micromolar dissociation constants, so that, at the concentrations used, the protein was saturated in both samples. Therefore, the resultant chemical shift differences originate solely from the small perturbation introduced by binding slightly different ligands. Similarly, resonance assignments were obtained for residues T104 and T103 with differential chemical shifts comparing spectra obtained for complexes formed with NADH and 3-acetyl pyridine NADH.

Resonance assignments were also obtained based on observation of protein-ligand NOEs. For a sample containing a complex of MIT-DHPR bound to NADH, the NADH ligand was perturbed through either a selective inversion (transient NOE) or complete saturation (steady-state NOE) of its resonances using radio-frequency pulses. These NOEs in the MITODHPR spectrum were observed in a 2D (¹H,¹H) NOESY spectrum (Anil-Kumar et al., supra (1980)). A portion of a 2D (¹H,¹H) NOESY spectrum of MIT-DHPR in complex with the cofactor NADH and the substrate analog PDC is shown in FIG. 1E. Due to the selective labeling scheme, little overlap was observed between the protein methyl-proton resonances and the ligand-proton resonances as shown in FIG. 1B. The resonance assignments of the NADH and PDC ligands were obtained from conventional 1D and 2D NMR experiments.

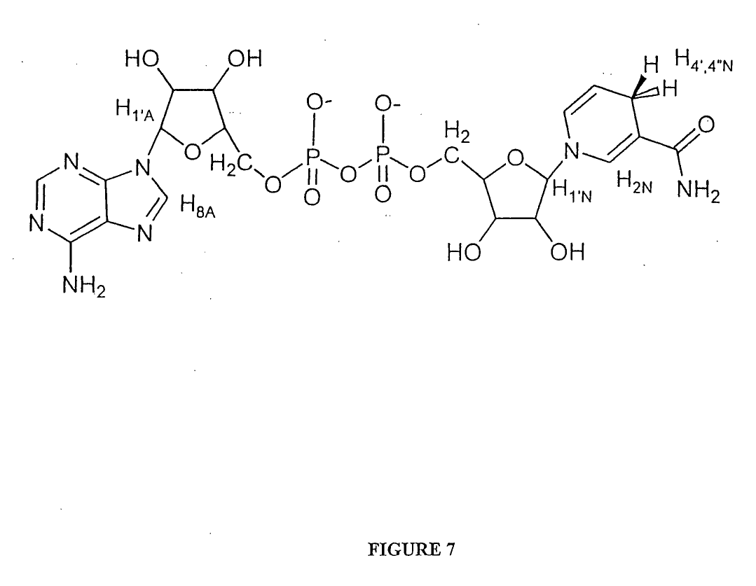

NOEs from the NADH reference ligand to protein atoms were interpreted in light of the existing crystal structure of the complex between DHPR, NADH and PDC (Scopin et al., supra (1997) , FIG. 1A). As shown in FIG. 1E, NOEs were observed between the H_{1′A} on NADH and Thr80, as well as between H_{2N} on NADH and Thr104 (see FIG. 7 for NADH atom designations). NOEs were also observed between Met17 and the pros H_{4′,4″N} proton. Thus, Thr80, Thr104 and Met17 were identified as key binding site residues. The above three assignments were based, in part, on the observation that in the crystal structure, Thr80, Thr104 and Met17 are the methyl containing amino acids that are closest to the atoms of NADH that are involved in the NOE (see FIG. 1A). It was possible to chirally assign the pros proton of the H_{4′,4″N} pair of protons as being proximal to Met17 based on the crystal structure, since it is known that the proR hydrogen is directed towards PDC, and the Met17 resides on the face of the nicotinamide ring opposite the PDC. NOEs were also observed between the PDC protons and the H_{4′,4″N} protons of NADH.

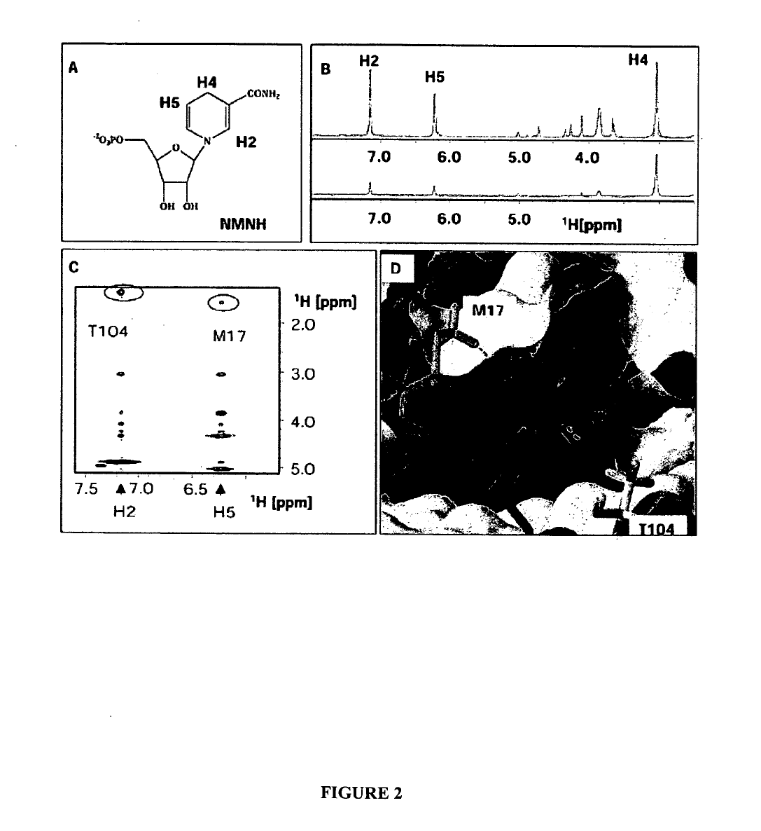

A complex was also formed between MIT-DHPR and nicotinamide mononucleotide (NMNH, FIG. 2A). The samples contained a low concentration of MIT-DHPR (0.01 mM) and 1 mM of NMNH. The resonances for the binding site-localized Met and Thr residues were saturated through saturation of the aliphatic region of the spectrum. The difference spectrum shown in FIG. 2B indicates that saturation was only transferred to NMNH when it was bound to MIT-DHPR.

The assignments for M17 and T104 were confirmed as follows. Strong inter-molecular NOEs between the nicotinamide ring protons and methyl groups of M17 and T104 were observed as shown in FIG. 2C. These cross-peaks were in agreement with the X-ray crystal structure of the DHPR-NADH-PDC ternary complex as shown in FIG. 2D.

The assignments of residues Thr104 and Met17 were also confirmed by comparing 2D (¹³C,¹H) correlation spectra of native and mutant (T104S-DHPR and M17I-DHPR) proteins. The disappearance of cross-peaks assigned to Thr104 for the T104S-DHPR spectra and cross-peaks assigned to Met17 for M17I-DHPR indicated that the assignments were correct.

E. Obtaining NOE Constraints for a Furoic Acid Inhibitor

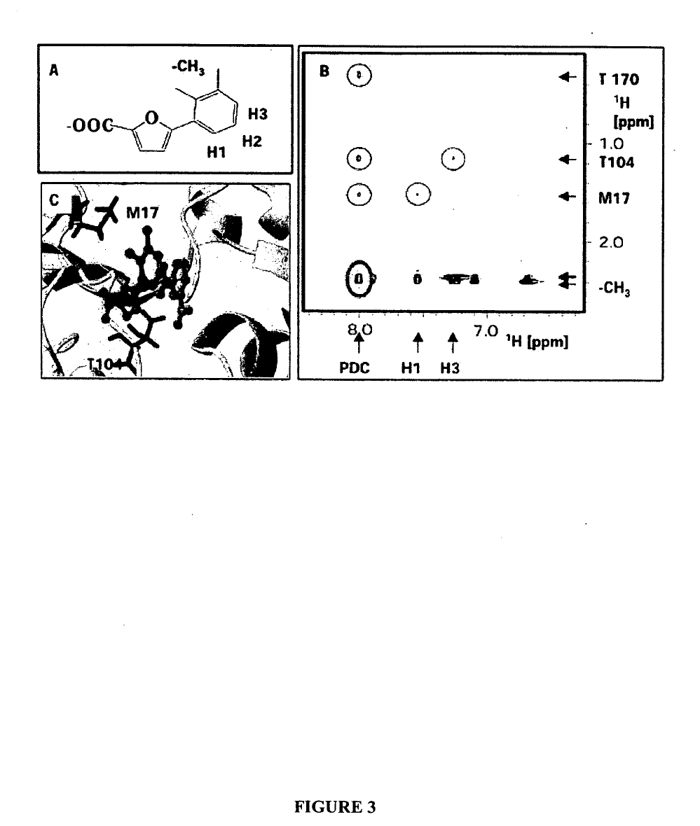

Distance constraints for the inhibitor TTM2000_{—}29_{—}85 (FIG. 3A) were obtained from NOESY measurements of the ternary complex formed by TTM2000_{—}29_{—}85, PDC and MIT-DHPR. As shown in FIG. 3B, NOEs were observed between PDC and protein Thr and Met methyl groups (circled in blue), between PDC and TTM2000_{—}29_{—}85 (circled in green) and between TTM2000_{—}29_{—}85 and protein (circled in red). Other NOEs not circled represent intra-molecular NOEs between the protons of the compound TTM2000_{—}29_{—}85. The NOEs between TTM2000_{—}29_{—}85 and protein and between TTM2000_{—}29_{—}85 and PDC were used as constraints in the docking simulations described below.

F. Docking of the Furoic Acid Inhibitor to DHPR

TTM2000_{—}29_{—}85 was docked into the binding site of the target enzyme based on the X-ray coordinates of DHPR when complexed with NADH and PDC (Scopin et al., supra (1997)), the NMR derived constraints with torsion angle dynamics as implemented in the software package DYANA (Guntert et al., J. Mol. Biol. 273:283-298 (1997)) and energy minimization of the resulting three-dimensional structures. During the docking simulations, the position of the PDC substrate analog and the coordinates of the enzyme were fixed and the NADH ligand was omitted. The coordinates of TTM2000_{—}29_{—}85 were obtained from the program InsightII (Molecular Simulation Inc., San Diego) and subsequently linked by a dummy linker of about 50 angstroms encompassing 80 dummy torsion angles. Random torsion angles were assigned to the linker in order to generate a model of the complex with random initial positioning of TTM2000_{—}29_{—}85. Subsequently, a variable target function was minimized in the linker torsion angle space in order to minimize the NOE distance constraints between TTM2000_{—}29_{—}85 and both protein and PDC. Twenty structures were calculated with 5000 iterations per structure. The best 7 structures converged into the final structure shown in FIG. 3C.

### EXAMPLE II

### Overlay of a Furoic Acid-Based Inhibitor onto DHPR-Bound NADH

This Example describes determination of a three dimensional model of a furoic acid-based inhibitor (TTM2000_{—}29_{—}85) by comparison to the structure of NADH when bound to E. coli Dihydrodipicolinate reductase (DHPR). In particular, this example describes comparing cross-peaks for a 2D NOESY spectrum of a DHPR-NADH complex with cross-peaks for a 2D NOESY spectra of a DHPR-TTM2000_{—}29_{—}85 complex and overlaying a structure model of TTM2000_{—}29_{—}85 and NADH based on distance constraints derived from the NOE measurements. As described below, neither assignment of DHPR-derived peaks to particular binding site residues nor a structural model of DHPR is necessary to determine structural properties of the inhibitor by ligand overlay.

DHPR is expressed, isotopically labeled and purified and NMR measurements are obtained as described in Example 1.

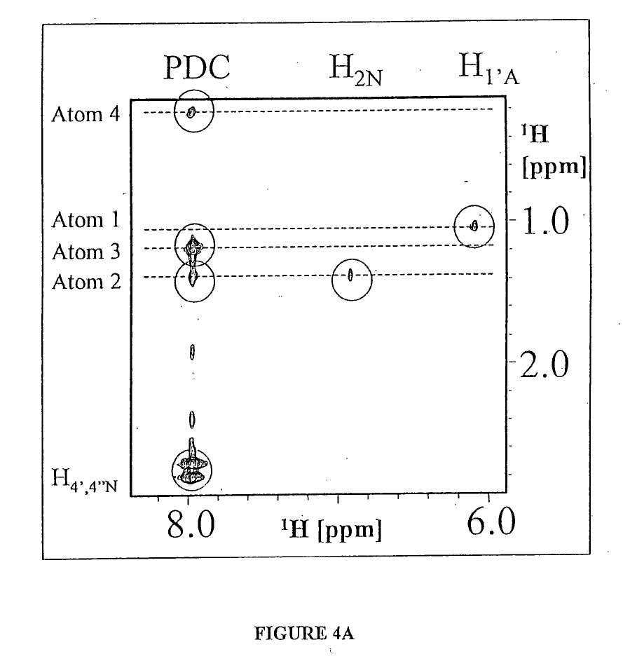

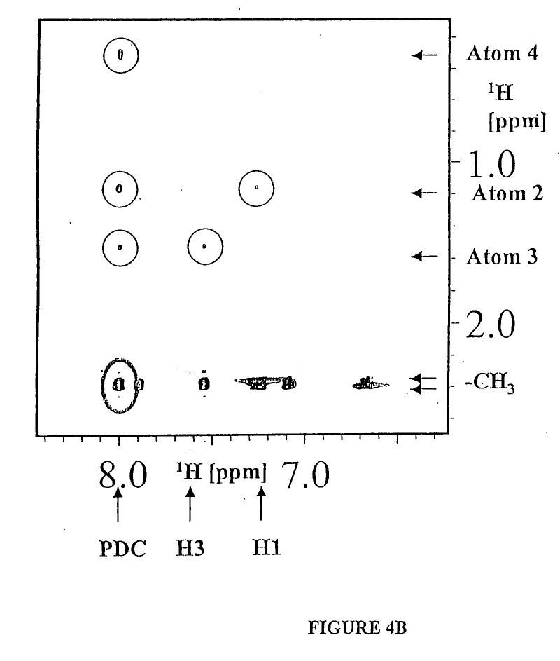

Binding site cross-peaks are identified from NOESY spectra for the ternary complex between PDC, NADH and DHPR having ¹³CH₃ labeled Threonine, Isoleucine and Methionine. NOEs are observed between H_{1′A} on NADH and an atom of DHPR identified as atom #1 (FIG. 4A), between H_{2N} on NADH and an atom of DHPR identified as atom #2 (FIG. 4A), and between H_{4′,4″N} and an atom of DHPR identified as atom #3. The above identifications are made according to relative proximity to atoms on the NADH reference ligand, without providing explicit amino acid assignments. NOEs are also observed between the PDC protons and the H_{4′,4″N} protons of NADH. Intramolecular NOEs are also observed for the NADH molecule, such as between H_{1′N} and H_{2N} indicating that the geometry around the nicotinamide glycosidic bond is anti, and between H_{1′A} and H_{8A} indicating that the geometry around the adenine glycosidic bond is anti (FIG. 7).

Similarly, NOESY spectra are obtained for the complex between TTM2000_{—}29_{—}85 and DHPR having ¹³CH₃ labeled Threonine, Isoleucine and Methionine. As shown in FIG. 4B, NOEs are observed between DHPR atom #2 and atom H1 of TTM2000_{—}29_{—}85, as well as between DHPR atom #3 and atom H3 of TTM2000_{—}29_{—}85 (see FIG. 3A for TTM2000_{—}29_{—}85 atom designations). Also, NOEs are observed between PDC protons and furoic acid methyl protons.

A structural model of TTM2000_{—}29_{—}85 is overlaid on the NADH molecule using the DGEOM software package (Quantum Chemistry Program. Exchange), with standard methods as described in the release of that software. The constraints between the reference ligand (NADH) and the test ligand (TTM2000_{—}29_{—}85) are derived for pairs of ligand atoms, one from each ligand, that have NOEs to a common protein atom. Accordingly, the following pairs of atoms are constrained to be within 3 angstroms of each other: (a) Furoic acid-H1 and NADH-H_{2N}, (b) Furoic acid-H3 and NADH-H_{4,′4″N}, and (c) Furoic acid-methyl protons and NADH-H_{4,′4″N}. NADH geometry is also constrained by the observed intramolecular NOEs. The geometry of NADH is allowed to vary in the calculation, however, its internal geometry can be fixed during the calculation based on its structure when bound to DHFR or by analogy with related structures of protein(s) with NADH bound.

### EXAMPLE III

### Validation of a Binding Site Homology Model for 1-Deoxy-D-Xylulose-5-Phosphate Reductoisomerase

This example demonstrates generation of a homology model for 1-Deoxy-D-xylulose 5-phosphate reductoisomerase (DOXPR) based on sequence analysis. Validation of the model using nuclear magnetic resonance spectroscopy is also demonstrated.

1-Deoxy-D-xylulose 5-phosphate reductoisomerase (DOXPR) is an enzyme involved in isoprenoid biosynthesis, catalyzing the formation of 2-C-methyl-D-erythritol from 1-deoxy-D-xylulose 5-phosphate (Takahashi et al., Proc. Natl. Acad. Sci. USA 95:9879-9884 (1998)). The deoxyxylulose pathway, found in some bacteria, algae, plants and protozoa, is an alternate to the ubiquitous mevalonate pathway for isoprenoid biosynthesis (Eisenreich et al., Trends Plant Sci. 6:78-84 (2001)). Because a three dimensional model of the DOXPR structure was not available and to aid in the design of inhibitors of DOXPR, a model for the NADPH-binding, N-terminal domain of the enzyme for E. coli was produced and validated as set forth below.

The E. coli DOXPR amino acid sequence was used to search for homologs with BLAST and PSI-BLAST using default parameters. Neither algorithm identified homologous sequences below an E-score of 0.005 in the Swiss-Prot database (other than orthologues of DOXPR). Other methods such as SDSC1 (Shindyalov and Bourne, Fourth Meeting on the Critical Assessment of Techniques for Protein Structure Prediction, A-92 (2000)) and 3D-JIGSAW (Bates and Sternberg, Proteins: Structure, Function and Genetics Suppl. 3:47-54 (1999)) were also unable to identify homologues for potential use as templates. The threading server 3D-PSSM (Kelley et al., J. Mol. Biol. 299:499-520 (2000)), also did not identify any hits below a significant E-value.

Homologs of E. coli DOXPR were identified from the Swiss-Prot database as follows. A search of the Swiss-Prot Database identified a set of 4,613 sequences for polypeptides that utilize NAD(P) to perform their enzymatic functions, including 28 DOXPR sequences. A comparison matrix was calculated for the set of sequences by characterizing each sequence by a string of scores that described its sequence similarity to every other sequence in the set. Each score was a percent identity score that was computed using BLAST 2.1.2 from NCBI as described in Nicholas et al., Biotechniques 28:1174-1191 (2000). The Euclidian distance between each of the sequence comparison signatures were measured as described in Manley,Multivariate Statistical Methods, a Primer, Chapman Hall 1994. Groups among the 4,613 sequences were defined using a divisive hierarchical clustering algorithm as described in Kaufman and Rousseeuw, Finding Groups in Data: An introduction to Cluster Analysis John Wiley and Sons, New York (1990). Cluster analysis using sequence identity scores yielded 94 sequence groups.

The 28 DOXPR sequences formed one cluster. When visualized in a comparison matrix, the DOXPR cluster was proximal to other clusters. These other clusters were composed of aspartate semialdehyde dehydrogenase, homoserine dehydrogenase, N-acetyl-g-glutamyl phosphate reductoisomerase, or glyceraldehyde 3-phosphate dehydrogenase; all of which share a common NAD(P)-binding Rossmann fold. The proximity correlated with local sequence identity between DOXPR sequences and sequences of these other clusters, ranging from about 17 to 40% local sequence identity. Although the E-scores of these sequence identities were between 0.1 and 2.0, these clusters were identified as related groups because multiple DOXPR sequences systematically showed cross-talk to only the above mentioned sequence clusters. In particular, cross-talk was identified as low sequence identity (less than 30%) between the cluster containing DOXPR and a few sequences belonging to other clusters, which showed a pattern that was distinct from a pattern observed in the cluster. The cross talk was distinguishable from true noise because in the case of noise, only a single DOXPR sequence had low similarity to some other cluster. Based on these data, the NADP-binding domain of E. coli DOXPR was predicted to contain a Rossmann fold.

The local sequence identities between the sequences in the proximal clusters occurred in the N-terminal, NAD(P)-binding domain. In order to choose a template for homology modeling of the DOXPR NAD(P)-binding domain, the sequences in the other clusters were evaluated according to their proximity to DOXPR in the sequence comparison matrix and whether or not a structural model was available for members of the cluster. Homoserine dehydrogenase and aspartate semialdehyde dehydrogenase showed the most proximity to DOXPR in the sequence comparison matrix. Of these two, a crystal structure was available for homoserine dehydrogenase.

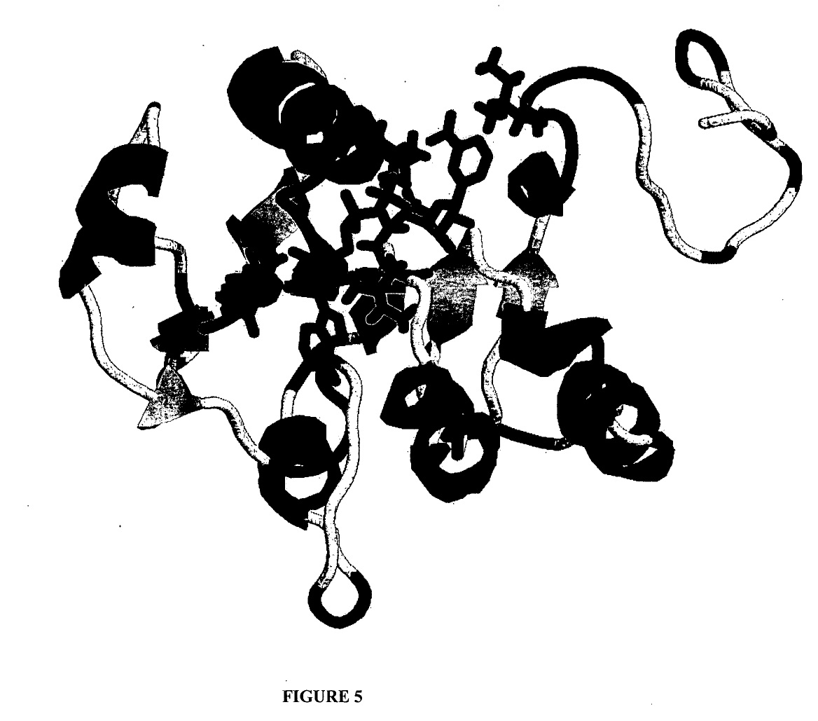

A multiple-alignment of E. coli DOXPR with the NAD-binding domain of S. cerevisiae homoserine dehydrogenase was performed using Clustalw (Thompson et al., Nucl. Acids. Res. 22:4673-4680 (1994)). The NAD-binding motif of E. coli DOXPR aligned very well with the NAD-binding motif of S. cerevisiae homoserine dehydrogenase. This alignment was used to build several models of E. coli DOXPR using the MODELER module in MSI Insight II (Sali and Blundell, J. Mol. Biol. 234:779-815 (1993)). The model having the least coiling of loops was chosen and is shown in FIG. 5, with some NADP-contact residues colored in blue (isoleucine), black (methionine), and cyan (lysine). The bound conformation of NAD from homoserine dehydrogenase is superimposed on the model and shown in green.

The validity of the homology model was tested using nuclear magnetic resonance (NMR) spectroscopy. Recombinant DOXPR was expressed under conditions for selective labeling with ¹³C^{ε}/¹H Met , ¹³C⁶⁷ /¹H Ile and ¹³C/¹H Thr and uniform labeling with ²H as described in Example I. MIT labeling was chosen based on a survey of oxidoreductase three-dimensional structures that revealed an average of four to five of these residues in the NAD-binding sites. MIT-DOXPR was purified as described in Meininger et al., Biochem. 39:26-36 (2000). For NMR measurements, MIT-DOXPR was at a concentration of 75 micromolar (300 micromolar monomer), pH=7.5 and T=303° K. ¹³C, ¹H correlation spectra were obtained with a 2D HMQC sequence as described in Example I with the exception that the selective WURST ¹³C homonuclear decoupling was applied at 27 ppm to decouple Ile ¹³C^{δ} (resonating at about 10 ppm) from Ile ¹³C^{Υ} (resonating at about 27 ppm). Typically, each 2D (¹³C,¹H) spectrum was recorded in about 30 minutes.

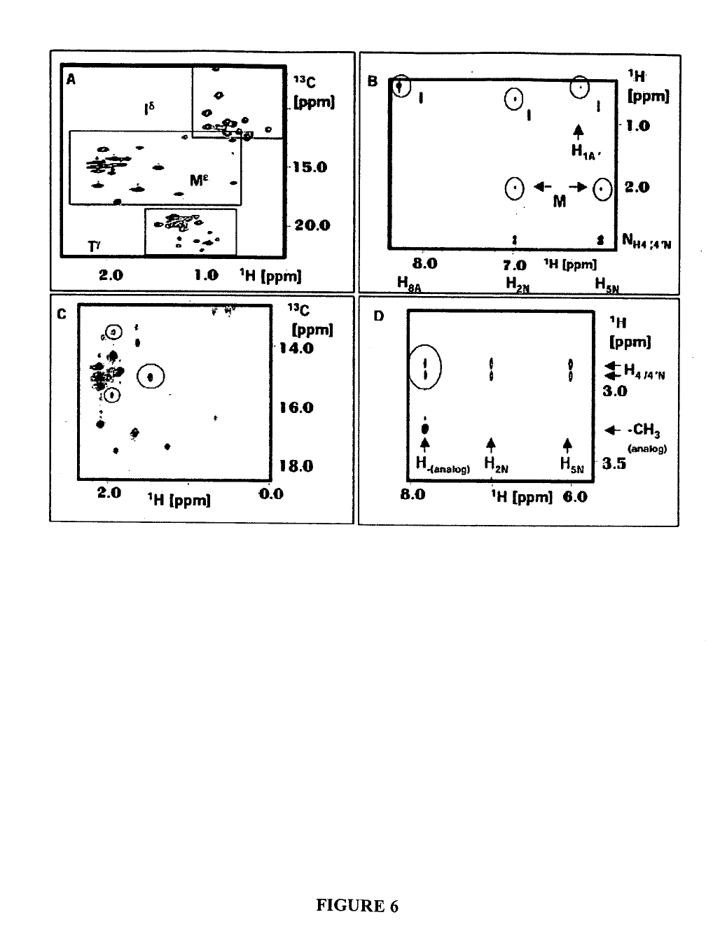

Based on proton chemical shifts, it was possible to observe changes in the chemical environment around NADPH and thereby determine which types of residues in the protein were interacting with the coenzyme. FIG. 6A shows a 2D (¹³C,¹H) HMQC spectrum for MIT-DOXPR. Met, Ile and Thr regions are enclosed in rectangles. NOE peaks observed between NADPH and residues in the binding pocket of E. coli DOXPR were consistent with those in the homology model in that a methionine and isoleucine were determined to be in proximity of the cofactor, with clear NOEs observed between the H_{2N} of NADPH and a Methionine as well as an Isoleucine as shown in FIG. 6B. The HSN atom of NADPH also showed an NOE to a Met residue (FIG. 6B). These observations were consistent with the homology model that had been constructed, which had Met 98 and Ile 13 in proximity to H2N of NADPH. The H1A′ and H8A protons of NADPH showed an NOE to a residue with proton chemical shifts typical for Isoleucines (FIG. 6B), and this is also consistent with the homology modeled structure for the NADPH-DOXPR binary complex, which has Ile 101 proximal to the H8A and H1A′ atoms of NADPH. Furthermore, the proximity of a lysine to the phosphate of NADPH is consistent with expectations. Thus, the model satisfied the constraints observed by NMR spectroscopy.

These results indicate that distance constraints derived from measurement of NMR interactions between a macromolecule and bound ligand can be used to confirm a theoretically based structure model. Such methods can also be used to drive the calculation of a homology model if the distance constraints are used in the modeling and docking process directly.

### EXAMPLE IV

### Identifying a Residue of DOXPR that is at an Interface between Ligand Binding Sites

This example demonstrates identification of a methionine residue in DOXPR that interacts with ligands bound to both the NADH binding site and substrate binding site. This example further describes construction of a bi-ligand combinatorial library based on identification of binding site-localized residues in combination with NMR-SOLVE methods.

The MIT-DOXPR protein was expressed, purified and NMR spectra obtained as described in Example III. DOXPR was determined to have a methionine and isoleucine in proximity of the NADPH cofactor as described in Example III.

Identification of active-site residues of metal binding proteins can be achieved through detection of line broadening using a paramagnetic metal ion probe. It has recently been proposed that DOXPR binds a Mn²⁺ ion with a catalytic role (Kuzuyama et al., 2000). 2D (13C,1H) HMQC spectra were acquired for MIT-DOXPR in the presence and absence of 10 micromolar Mn²⁺. Comparison of the spectra indicated three Met residues having atoms that interacted with Mn²⁺ (FIG. 6C). One of the Met residues also exhibited NOEs with the cofactor NADPH, therefore further indicating that the Met was positioned at the interface between the cofactor and substrate binding sites.

In the case of DOXPR, for which a crystal structure was not available, the location of the interface Met residue in the primary sequence was not unambiguously identified. However the chemical shift of the binding site-localized Met was identified. Detection of NOEs between a candidate inhibitor and the met having a resonance at this chemical shift location provides information about the orientation of the inhibitor. relative to the NADPH cofactor. Assignment of the atom of the inhibitor that interacts with the Met residue indicates that this atom or others proximal to it are a location for a linker connecting the inhibitor to NADPH-mimics for formation of a bi-ligand inhibitor. Thus, NMR-SOLVE is used to guide bi-ligand combinatorial library construction without knowledge of the three-dimensional structure of the DOXPR target.

Inter-ligand NOEs in DOXPR between a stable version of an enolate intermediate analog, that binds to DOXPR with a Kᵢ of 470 micromolar, and the cofactor NADPH were observed (FIG. 6D). These inter-ligand NOEs are used to identify molecules that bind in the catalytic portion of the cofactor binding site of the enzyme, and to determine their orientation relative to the substrate binding pocket.

Throughout this application various publications, patents and patent applications have been referenced. The disclosures of these publications, patents and patent applications in their entireties are hereby incorporated by reference in this application in order to more fully describe the state of the art to which this invention pertains.

The term “comprising” is intended herein to be open-ended, including not only the recited elements, but further encompassing any additional elements.

Although the invention has been described with reference to the examples provided above, it should be understood that various modifications can be made without departing from the spirit of the invention. Accordingly, the invention is limited only by the claims.

1-26. (canceled)

27. method for determining a structure model for a test ligand bound to a macromolecule binding site, wherein a reference complex can be formed between the macromolecule binding site and a reference ligand, and wherein a test complex can be formed between the macromolecule binding site and a test ligand, comprising the steps of:
(a) providing a structure model of the reference ligand bound to the macromolecule binding site;
(b) observing NMR signals for the reference complex, wherein NMR signals for reference ligand atoms interact with signals for atoms of the macromolecule;
(c) assigning NMR signals to the reference ligand atoms that interact with the atoms of the macromolecule in the reference complex;
(d) identifying NMR signals for atoms of the macromolecule that interact with the assigned NMR signals for the reference ligand atoms;
(e) selectively observing pairs of interacting NMR signals for the test complex, each pair comprising an NMR signal for the test ligand that interacts with an NMR signal for an atom of the macromolecule identified in part (d), thereby identifying test ligand atoms and reference ligand atoms that interact with a common macromolecule atom; and
(f) overlaying a structure model of the test ligand on the structure model of the reference ligand, wherein atoms for the test ligand and reference ligand that interact with a common macromolecule atom are overlapped,
thereby determining a structure model for the test ligand bound to the macromolecule binding site.

28. The method of claim 27, wherein the macromolecule is isotopically labeled.

29. The method of claim 27, wherein the macromolecule comprises a polypeptide.

30. The method of claim 29, wherein the polypeptide is isotopically labeled with an atom selected from the group consisting of ²H, ¹⁵N and ¹³C.

31. The method of claim 29, wherein the polypeptide is isotopically labeled at a backbone position.

32. The method of claim 29, wherein the polypeptide is isotopically labeled at a side-chain position.

33. The method of claim 32, wherein the side chain position comprises a methyl position of an amino acid selected from the group consisting of methionine, leucine, isoleucine, threonine, alanine and valine.

34. The method of claim 29, wherein the type of amino acid that contains the common macromolecule atom is identified.

35. The method of claim 29, wherein the position and type of amino acid that contains the common macromolecule atom is identified.

36. The method of claim 27, wherein step (g) further comprises performing an energy-minimization refinement of the structure model for the test ligand, the structure model for the reference ligand or both.

37. The method of claim 27, wherein step (g) further comprises performing a molecular dynamics simulation refinement of the structure model for the test ligand, the structure model for the reference ligand or both.

38. The method of claim 27, wherein the macromolecule has a monomeric molecular weight that is at least 25 kDa.

39. The method of claim 27, wherein less than 70% of the atoms of the macromolecule are assigned an NMR signal.

40. The method of claim 27, wherein the interacting NMR signals comprise cross-peaks in a two-dimensional NMR spectrum.

41. The method of claim 27, wherein the interacting signals interact due to a Nuclear Overhauser Effect, chemical shift perturbation, or relaxation effect.

42. The method of claim 27, wherein the NMR signals are detected by a double-resonance method.

43. The method of claim 42, wherein the double-resonance method is selected from the group consisting of COSY, HMQC, HSQC and NOESY.

44. The method of claim 27, wherein the NMR signals are detected by a triple-resonance method.

45. The method of claim 44, wherein the triple-resonance method is selected from the group consisting of HNCA, HNCO, HNCACB, CBCA(CO)NH, HBHA(CO)CA, HN(CO)CA, H(CA)NH, H(CC) {TOCSY}NH, and heteronuclear resolved NOESY.

46. The method of claim 27, wherein the NMR signals are detected using a TROSY pulse sequence.

47. The method of claim 46, wherein the NMR signals are detected using a SEA-TROSY pulse sequence.

48. The method of claim 27, further comprising providing a structure model of the macromolecule binding site.

49. The method of claim 48, wherein step (f) further comprises docking a structure model of the test ligand to the structure model of the macromolecule binding site.

50. The method of claim 48, wherein the structure model of the macromolecule binding site is selected from the group consisting of an X-ray crystal structure model, an NMR structure model and a theoretical structure model.

51-70. (canceled)
